(19) United States
(12) Patent Application Publication
(10) Pub. No.: US 2004/0241164 A1
(43) Pub. Date: Dec. 2, 2004

(54) Use of antibodies having high affinity for soluble ab to treat conditions and diseases related to ass

(76) Inventors: Kelly Renee Bales, Indianapolis, IN (US); Steven Marc Paul, Carmel, IN (US)

Correspondence Address: ELI LILLY AND COMPANY, PATENT DIVISION, P.O. BOX 6288, INDIANAPOLIS, IN, 46206-6288, US

(21) Appl. No.: 10/487,326

(22) PCT Filed: Aug. 14, 2002
(86) PCT No.: PCT/US02/21324

Related U.S. Application Data
(60) Provisional application No. 60/313,576, filed on Aug. 17, 2001.
(60) Provisional application No. 60/383,851, filed on May 28, 2002.

(51) Int. Cl. A61K 39/395

(52) U.S. Cl. 424/145.1

(57) ABSTRACT

The invention provides a method for treating a subject having a condition or disease related to the Aβ peptide comprising administering to the subject an antibody that recognizes Aβ, wherein the antibody has an affinity for soluble Aβ higher than 10⁻⁹M.

This application claims the priority of U.S. Provisional Application 60/313,576, filed Aug. 17, 2001, and U.S. Provisional Application 60/383,851, filed May 28, 2002, both of which are expressly incorporated by reference.

This invention is in the field of medicine. More particularly, this invention is in the field of treatment of conditions and diseases related to the Aβ peptide, such as Alzheimer's disease, Down's syndrome, cerebral amyloid angiopathy, mild cognitive impairment, and the like.

Chronic administration (several months) of antibodies that recognize certain N-terminal epitopes within Aβ (e.g., polyclonal antibodies raised against aggregated Aβ1-42 and monoclonal antibodies 3D6 and 10D5) reduced amyloid plaque in the brains of PDAPP mice [Bard, et al. Nature Med. 6:916-919 (2000); Schenk, et al. WO00/72880, Dec. 7, 2000]. Schenk found that efficacy in reducing plaque was not related to antibody affinity for aggregated Aβ, despite stating a preference for antibodies with affinity for Aβ greater than 10⁶ M⁻¹, 10⁷ M⁻¹, 10⁸ M⁻¹, 10⁹ M⁻¹, or 10¹⁰ M⁻¹. These affinities must be in terms of affinity for aggregated Aβ because; 1) the affinities for aggregated Aβ are the only affinities specifically mentioned in the disclosure for specific antibodies; 2) the disclosure reported active immunization that relied on aggregated Aβ for vaccinations; 3) the disclosure reported that Aβ plaque reduction following passive immunization with polyclonal antibodies raised against aggregated Aβ or with other anti-Aβ antibodies requires adherence of the antibodies to aggregated Aβ in plaques followed by cell mediated plaque clearance.

Several studies have shown that administration of aggregated Aβ improves performance in various tests of memory and cognition in animal models of Alzheimer's disease after chronic administration (several weeks to several months) [Weiner, et al., Ann. Neurol. 48:567-579 (2000); Janus, et al., Nature 408:979-982 (2000); Morgan, et al., Nature 408:982-985 (2000)]. However, these investigators used aggregated Aβ, and so presumably obtained polyclonal antibodies against aggregated Aβ. Such treatment, apparently somewhat effective to improve cognition, is very prolonged and inefficient. Furthermore, the use of forms of Aβ as antigens may be hazardous and produce immunological responses that are difficult to control and quite variable between subjects.

DeMattos, et al. [Proc. Nat'l Acad. Sci. (USA) 98:8850-8855 (2001)] identified an Aβ-antibody complex after administration of antibodies 266 [Seubert, et al., Nature 359:325-327 (1992)] or 4G8 [Shoji, et al., Science 258:126-129 (1992)], antibodies that recognize epitopes between amino acids 13 and 28 of Aβ. No complexes were observed when antibodies that recognize other portions of Aβ were administered, even though some had affinities for soluble Aβ similar to that of 4G8. It was concluded that antibodies recognizing epitopes in the 13-28 region of Aβ would cause formation of the complex and sequester Aβ in the plasma. The affinity of the antibody for Aβ was not considered an important factor in sequestration of Aβ in plasma. DeMattos also showed that administration of 266 causes a rapid, massive efflux of Aβ from the CNS to the plasma within hours after administration.

We have now found that the rate of Aβ flux in the first 24 hours after administration of antibodies is related to the affinity of the antibody for soluble Aβ, and that antibodies with higher affinity for soluble Aβ cause a more rapid efflux of soluble Aβ. Most surprisingly, rapid recovery of cognitive performance was seen within 24 hours of administering certain antibodies. The relative magnitude of recovery was related to the rate of efflux of soluble Aβ within the first 24 hours, and to the affinity of the antibody for soluble Aβ.

The invention proceeds from the first recognition of an important feature of anti-Aβ antibodies that was previously unrecognized and could not have been deduced from the prior art, which in fact taught that the important features of effective anti-Aβ antibodies were either the particular epitope recognized (e.g., N-terminal) or the affinity of the antibody for aggregated forms of Aβ. The newly recognized, important feature of anti-Aβ antibodies is their affinity for soluble forms of Aβ. An antibody having a higher affinity for soluble Aβ will be more effective in treating conditions or diseases related to the Aβ peptide.

### SUMMARY OF THE INVENTION

Therefore, this invention provides a method for treating a subject having a condition or disease related to the Aβ peptide comprising administering to the subject an antibody that recognizes Aβ, wherein the antibody has greater affinity for soluble Aβ than 10⁻⁹ M. More particularly, the invention is a method for treating cognitive symptoms of a condition or disease associated with Aβ in a subject, comprising administering to the subject an effective amount of an anti-Aβ antibody that has greater affinity for soluble Aβ than antibody 266 has. More particularly, the affinity is with respect to Aβ1-40 or Aβ1-42.

The invention includes a method for reducing disease progression in a subject having a condition or disease associated with Aβ, comprising administering to the subject an effective amount of an anti-Aβ antibody that has greater affinity for soluble Aβ than 10⁻⁹ M. More particularly, the invention is a method for reducing disease progression in a subject having a condition or disease associated with Aβ, comprising administering to the subject an effective amount of an anti-Aβ antibody that has greater affinity for soluble Aβ than antibody 266 has. More particularly, the affinity is with respect to Aβ 1-40 or Aβ 1-42.

The invention also includes a method for treating cognitive symptoms of a condition or disease associated with Aβ in a subject, comprising administering to the subject an effective amount of an anti-Aβ antibody that has affinity for soluble Aβ1-40 or Aβ1-42 higher than 10⁻⁹ M, more particularly, higher than the affinity of antibody 266.

Also included is the use of an anti-Aβ antibody that has affinity for soluble Aβ1-40 or Aβ1-42 higher than 10⁻⁹ M (i.e., higher than the affinity of antibody 266) for preparing medicaments for reducing disease progression or treating cognitive symptoms of a condition or disease associated with Aβ. More particularly, the invention is the use of an anti-Aβ antibody having higher affinity for soluble Aβ than antibody 266 has to prepare a medicament for treating cognitive symptoms or reducing disease progression in a subject having a condition or disease associated with Aβ.

### BRIEF DESCRIPTION OF THE DRAWINGS

FIG. 1. Object recognition memory performance 24 hours after administration of m266 anti-Aβ antibody. The recognition index is the percentage of time spent exploring a novel object during trial 2 (test trial). Both saline- and control IgG-treated tg mice performed at chance levels (recognition index=50%), whereas m266-treated tg mice and WT mice significantly performed above chance (t-test analysis). Values are means±SEM; ** means p<0.0001 vs. saline- and IgG-treated tg groups; ## means p<0.0001 vs. wild type (WT) mice.

FIG. 2. Plasma Aβ40 and Aβ42 levels 24 hours after administration of m266. Plasma levels correlated with object recognition memory performance. (A) Plasma levels of both peptides are markedly increased in APP^{V717F} tg mice acutely administered m266, compared to saline or control IgG-treated tg mice. Values are means ±SEM; (B) Bivariate scattergrams showing highly significant correlation between plasma levels of Aβ and the object recognition memory performance.

FIG. 3. Apparatus used for holeboard spatial learning assay.

FIG. 4. Acute Aβ antibody treatment improved reference memory in APPV717F mice.

FIG. 5. Acute Aβ antibody treatment decreased total errors in APPV717F mice.

FIG. 6. Correlation between Log (Aβ flux) and Log (affinity of various anti-Aβ antibodies for soluble Aβ).

FIG. 7. Lack of correlation between Log (Aβ flux) and Log (affinity of various anti-Aβ antibodies for insoluble Aβ).

FIG. 8. Object recognition memory performance 24 hours after administration of 266 or 3D6 anti-Aβ antibody. (* means p<0.05 vs. saline or IgG, *** means p<0.001 vs. saline or IgG).

FIG. 9. Correlation between Log (Aβ flux) and Log (affinity of various anti-Aβ antibodies for soluble Aβ using altered BIAcore method).

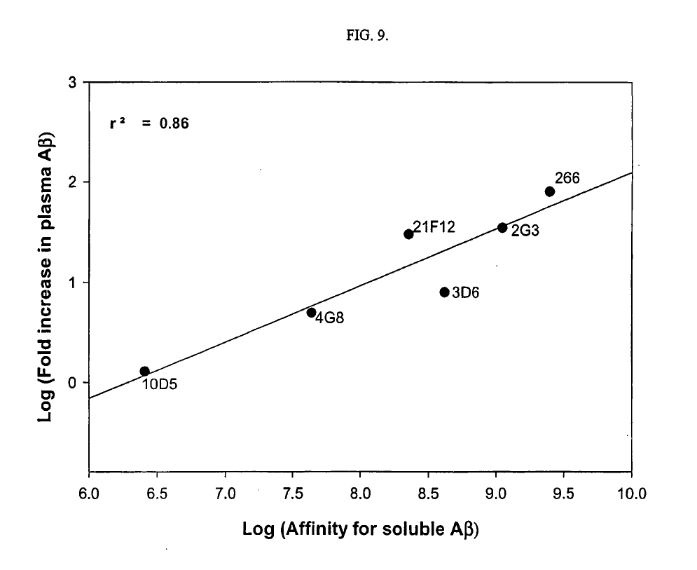

### DETAILED DESCRIPTION OF THE INVENTION

By “cognition” is meant short-term memory, long-term memory, abstraction, judgment, language, praxis, visuospatial skills, behavior or personality. Cognition may be assessed in non-human subjects using any of a wide array of tests [Weiner, et al., Ann. Neurol. 48:567-579 (2000); Janus, et al., Nature 408:979-982. (2000); Morgan, et al., Nature 408:982-985 (2000); Dodart, et al., Neuroreport. 8:1173-1178 (1997)], and the methods described herein. Cognition in humans may be assessed using any of the tests mentioned below, plus tests such as the Alzheimer's Disease Assessment Scale-Cognitive subscale (ADAS-Cog) component [Rosen, et al., Am J Psychiatry 141: 1356-1364 (1984)]. The ADAS-Cog is a 70-point test that briefly assesses memory, language capabilities, and other cognitive functions in patients with dementia.

By “subject” is meant a mammal, preferably a human. A subject will benefit from the present invention if the subject has a cognitive deficiency or aberration caused by or related to the presence of toxic forms and/or concentrations of soluble Aβ in the subject's brain. Even though the nature or concentration of Aβ in a subject's brain may not be known with certainty, the administration of well-known tests of cognition in subjects who are suspected or known to suffer from an Aβ-related disease will suffice to identify many subjects who will benefit from the present methods. For other subjects, a combination of clinical assessment, subject history, and perhaps laboratory or other diagnostic assessments may be needed to identify subjects likely to benefit from the present invention.

A mental status examination of cognitive domains such as language, memory, visuospatial function, executive function (ability to manipulate previously acquired information, multitasking, abstraction, judgment, calculation, etc.), personality, and mood will aid in identifying subjects most likely to benefit from the present invention. Subjects meeting the criteria for a diagnosis of probable AD (i.e., dementia, 40-90 years old, cognitive deficits in two or more cognitive domains, progression of deficits for more than six months, consciousness undisturbed, and absence of other reasonable diagnoses) will benefit from the present invention. Likewise, subjects with Down's syndrome will benefit from the present invention.

The Mini-Mental State Examination (MMSE) is widely used, with norms adjusted for age and education [Folstein et al, J. Psych. Res. 12:196-198 (1975); Anthony, et al., Psychological Med. 12: 397-408 (1982); Cockrell, et al., Psychopharmacology 24: 689-692 (1988); Crum, et al., J Am. Med. Assoc'n 18:2386-2391 (1993)]. The MMSE is a brief, quantitative measure of cognitive status in adults. It can be used to screen for cognitive impairment, to estimate the severity of cognitive impairment at a given point in time, to follow the course of cognitive changes in an individual over time, and to document an individual's response to treatment. Cognitive assessment of subjects may require formal neuropsychologic testing, with follow-up testing separated by nine months or more (in humans). The “cognitive symptoms” treated by the present invention are cognitive deficits known to be associated with conditions and diseases related to Aβ as discussed herein.

Laboratory assessment or structural imaging studies may identify reversible causes of cognitive impairment, which are not likely to respond to the present invention, and to identify focal lesions, significant white matter disease implicating vascular dementia, significant temporal atrophy. In AD, functional studies such as positron emission tomography (PET) or single-photon emission computed tomography (SPECT) typically show abnormalities most marked in the parietal and temporal lobes bilaterally. These studies are particularly useful, in differentiating early AD from normal aging or frontotemporal degeneration. They are not required to identify subjects who are likely to benefit from the present invention. The combination of medial temporal atrophy determined by structural imaging and parietal impairment determined with functional imaging is, however, a useful biomarker of AD. MRI can be used to exclude subjects with significant cerebrovascular disease.

ApoE genotyping is not useful in isolation, but may increase the specificity of the diagnosis when patients do not have the E4 allele if the diagnosis is in question. Another potential biomarker is the combined assessment of cerebral spinal fluid (CSF) Aβ 42 and tau concentrations. A low Aβ42 and high tau concentration have a high predictive value (90%) and negative predictive value (95%) based on a clinical diagnosis of probable AD.

By “condition or disease related to Aβ ” is meant conditions and diseases that are associated with: 1) the development of P-amyloid plaques in the brain, 2) the synthesis of abnormal forms of Aβ, 3) the formation of particularly toxic forms of Aβ, or 4) abnormal rates of synthesis, degradation, or clearance of Aβ. Conditions and diseases such as clinical and pre-clinical Alzheimer's disease, Down's syndrome, cerebral amyloid angiopathy, certain vascular dementias, and mild cognitive impairment are known or suspected of having relationship to Aβ. “Disease progression” refers to worsening of signs or symptoms of the condition or disease with time.

Alzheimer's disease, discussed above, is the most prevalent disease related to Aβ (60-80% of dementias). Definite diagnosis of AD is only possible presently with a post-mortem examination. But, a diagnosis of probable AD correlates highly with AD pathology. Vascular dementia (VaD), dementia with Lewy bodies (DLB), and frontotemporal dementia (FTD) together probably account for 15% to 20% of dementias, with other disorders (e.g., hydrocephalus; vitamin B12 deficiency) accounting for about 5%. Of these, only certain vascular dementias are suspected of having a significant Aβ component.

A state of increased risk or early manifestation of cognitive problems that often progresses to AD is termed mild cognitive impairment (MCI). MCI is a clinical entity characterized by memory loss, without significant dysfunction in other cognitive domains and without impairment in activities of daily living (ADL) function. Early diagnosis and treatment of MCI, including with the use of the present invention, is important. Currently the best predictor of preclinical AD is a diagnosis of MCI, because 30-50% of subjects with MCI develop AD within 3-5 years. One structural correlate of MCI that may be predictive for which subjects will develop AD is the volume of the hippocampus. Subjects with MCI have smaller hippocampi than age-equivalent controls and appear to experience atrophy of the structure at a more rapid pace.

By “administering” is meant the act of introducing a pharmaceutical agent into the subject's body. The parenteral route is the preferred route of administering the antibodies in the methods of the present invention. Preferred parenteral routes include subcutaneous, intravenous, and intraperitoneal.

By “effective dose” is meant an amount of antibody, which when administered to the subject, will cause improvement in cognition. The amount of antibody in an effective dose can be readily determined by a skilled physician or clinical pharmacologist, taking into account the subject's body mass, age, gender, severity of the Aβ-related condition or disease, affinity of the antibody for soluble Aβ, route of administration, and similar factors well known to physicians and pharmacologists. Effective doses may be expressed, for example, as the total mass of antibody (e.g., in grams, milligrams or micrograms) or as a ratio of mass of antibody to body mass (e.g., as grams per kilogram (g/kg), milligrams per kilogram (mg/kg), or micrograms per kilogram (μg/kg). An effective dose of antibody in the present methods will range between 1 μg/kg and 100 mg/kg. A more preferred range for effective dose in the present invention is between 1 μg/kg and 30 mg/kg. Yet more preferred ranges are between 1 μg/kg and 10 mg/kg, 1 μg/kg and 10 mg/kg, between 1 μg/kg and 1 mg/kg, between 1 μg/kg and 0.3 mg/kg, and between 1 μg/kg and 0.1 mg/kg.

“Aβ peptide” and “Aβ ” refer to a peptide that is derived from amyloid precursor protein (“APP”—Alzheimer's disease amyloid A4 protein [Precursor]) by proteolytic cleavage. Full-length Aβ peptides are from 39 to 43 amino acids long in humans, for example. Full length Aβ peptide may undergo further cleavage in vivo to produce Aβ fragments that are shorter at the N-terminus, at the C-terminus, or both, by one to several amino acids. Soluble full-length Aβ peptide or fragments thereof may be used as antigens to raise antibodies that bind soluble Aβ peptide with high specificity and affinity. For example, among the many Aβ peptide fragments used for this purpose, the Aβ 13-28 fragment (conjugated via m-maleimidobenzoyl-N-hydroxysuccinimide ester to an anti-CD3 antibody) was used to raise antibody 266 [Seubert, et al., Nature 359:325-327 (1992)]. The use of antibody 266 for selective measurement of soluble Aβ is well known [Schenk, et al., U.S. Pat. Nos. 5,593,846, 5,766,846, 5,872,672, and 6,284,221 B1]. Assessment of binding to “soluble Aβ ” is carried out with Aβ in an unaggregated form, predominantly monomeric form, as described hereinbelow.

The expression “anti-Aβ antibody” means an antibody that binds to soluble Aβ. The antibody preferably binds with high affinity to soluble Aβ. Affinity higher than that of antibody 266 is preferred. Affinity higher than 10⁻⁹ M is preferred. Affinity higher than 10⁻¹⁰ M is more preferred. Affinity higher than 10⁻¹¹ M is yet more preferred. Affinity higher than 10⁻¹² M is highly preferred. The term “Aβ ” in this context includes the 39, 40, 41, 42, and 43 amino acid peptides derived from the APP protein in vivo by proteolysis, and any fragments of those peptides, such as N-terminally shortened peptides derived from those peptides (e.g., denoted by, for example, x-42, where x=1, 2, 3, etc.), C-terminally shortened peptides derived from 1-39, 40, 41, and 42 peptides, and peptides shortened at both termini. The expression “Aβ 40” is used to denote peptides that bind to antibodies that bind only at an Aβ C-terminus that ends at position 40. The expression “Aβ 42” denotes peptides that bind to antibodies that bind only at an Aβ C-terminus that ends at position 42.

By “affinity” is meant the strength of the binding of a single antigen-combining site with an antigenic determinant. It is a measure of the binding strength between antibody and a simple hapten or antigen determinant. It depends on the closeness of stereochemical fit between antibody combining sites and antigen determinants, on the size of the area of contact between them, and on the distribution of charged and hydrophobic groups. It includes the concept of “avidity,” which refers to the strength of the antigen-antibody bond after formation of reversible complexes. The most direct way of measuring antibody affinity is by the well known method of equilibrium dialysis. Methods requiring less time or materials than equilibrium dialysis are known, for example, the method of Griswold, et al. Immunology Letters 9:15-18 (1985) and the kinetic BIAcore method described herein. The BIAcore method relies on the phenomenon of surface plasmon resonance (SPR), which occurs when surface plasmon waves are excited at a metal/liquid interface. Light is directed at, and reflected from, the side of the surface not in contact with sample, and SPR causes a reduction in the reflected light intensity at a specific combination of angle and wavelength. Bimolecular binding events cause changes in the refractive index at the surface layer, which are detected as changes in the SPR signal.

The dissociation constant, KD, and the association constant, KA, are quantitative measures of affinity. At equilibrium, free antigen (Ag) and free antibody (Ab) are in equilibrium with antigen-antibody complex (Ag-Ab), and the rate constants, ka and kd, quantitate the rates of the individual reactions:

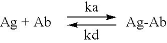

At equilibrium, ka [Ab][Ag]=kd [Ag−Ab]. The dissociation constant, KD, is given by: KD=kd/ka=[Ag][Ab]/[Ag−Ab]. KD has units of concentration, most typically M, mM, mM, nM, pM, etc. When comparing antibody affinities expressed as KD, having greater affinity for Aβ is indicated by a lower value. The association constant, KA, is given by: KA=ka/kd=[Ag−Ab]/[Ag][Ab]. KA has units of inverse concentration, most typically M⁻¹, mM⁻¹, μM⁻¹, nM⁻¹, pM⁻¹, or the like. When comparing antibody affinities expressed as KA, having greater affinity for Aβ is indicated by a higher value. “Affinity for soluble” Aβ is measured as described herein using samples of Aβ, typically Aβ1-40 or Aβ1-42, that are reasonably free of aggregated forms of Aβ. For antibodies having high affinity for soluble Aβ, particular care must be taken when using the BIAcore technology, as described hereinbelow.

As used herein, the word “treat” includes therapeutic treatment, where a condition to be treated is already known to be present, and prophylaxis—i.e., prevention of, or amelioration of, the possible future onset of a condition. The term “treating” includes prophylaxis (preventing), amelioration (reducing or reversing), or elimination of a sign, symptom, condition, disease, or disorder.

By “antibody” is meant a whole antibody, including without limitation an animal-derived antibody (e.g., murine), chimeric, humanized, human sequence, recombinant, transgenic, grafted and single chain antibody, and the like, and any fusion proteins, conjugates, fragments, or derivatives thereof. An antibody comprises protein resembling an antibody in the broadest sense in that the protein comprises a binding site for an antigen, which binding site is comprised of three pairs of complementarity determining regions. Antibody includes a whole immunoglobulin molecule, a monoclonal antibody, a chimeric antibody, a humanized antibody, a human antibody, or an immunologically effective fragment of any of these. An antibody fragment, or simply fragment, means an Fv, a disulfide linked Fv, scFv, Fab, Fab′, or F(ab′)₂ fragment, which terms are well known in the art. In some contexts herein, fragments will be mentioned specifically for emphasis. Nevertheless, it will be understood that regardless of whether fragments are specified, the term “antibody” includes such fragments as well as single-chain forms. As long as a protein retains the ability specifically to bind its intended target, it is included within the term “antibody.” Also included within the definition “antibody” are single chain forms. Preferably, but not necessarily, the antibodies useful in the invention are produced recombinantly. Antibodies may or may not be glycosylated, though glycosylated antibodies are preferred under some circumstances, such as when prolonged residence in the body is desirable, or when minimum risk of developing neutralizing antibodies. Antibodies, except perhaps for certain types in which cross-linking between chains is accomplished by peptide or other chemical chains, are properly cross-linked via disulfide bonds.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Light chains are classified as kappa and lambda. Heavy chains are classified as gamma, mu, alpha, delta, or epsilon, and define the antibody's isotype as IgG, IgM, IgA, IgD and IgE, respectively. IgG isotypes are preferred. Of the IgG subclasses, IgG1 and IgG4 are preferred.

The variable regions of each light/heavy chain pair form the antibody binding site. Thus, an intact non-fragment antibody and certain fragments (e.g., an F(ab′)₂ fragment) has two binding sites, whereas, most fragments have only one binding site per molecule. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with well known conventions [Kabat, et al., “Sequences of Proteins of Immunological Interest” National Institutes of Health, Bethesda, Md., 1987 and 1991; Chothia, et al., J. Mol. Biol. 196:901-917 (1987); Chothia, et al., Nature 342:878-883 (1989)].

By “humanized antibody” is meant an antibody that is composed partially or fully of amino acid sequences derived from a human antibody germline by altering the sequence of an antibody having non-human complementarity determining regions (CDR). Humanized antibodies are also referred to as CDR-grafted or reshaped antibodies. A humanized immunoglobulin does not encompass a chimeric antibody having a mouse variable region and a human constant region. However, the variable region of the antibody and even the CDR are humanized by techniques that are by now well known in the art. The framework regions of the variable regions are substituted by the corresponding human framework regions leaving the non-human CDR substantially intact. As mentioned above, it is sufficient for use in the methods of the invention, to employ an immunologically specific fragment of the antibody, including fragments representing single chain forms.

Although the mechanics of producing an engineered, humanized mAb using the techniques of molecular biology are relatively straightforward, simple grafting of xenogenic (usually rodent) complementarity-determining regions (CDRs) into human frameworks does not always reconstitute the binding affinity and specificity of the original mAb. The design of the engineered mAb is the critical step in reproducing the function of the original molecule. This design includes various choices: the extents of the CDRs, the human frameworks to use and the substitution of residues from the rodent mAb into the human framework regions (backmutations). The positions of these backmutations have been identified principally by sequence/structural analysis or by analysis of homology models of the variable regions' 3D structure. Recently, phage libraries have been used to vary the amino acids at chosen positions. Similarly, many approaches have been used to choose the most appropriate human frameworks in which to graft the rodent CDRs. Variable regions with high amino acid sequence identity to the rodent variable regions (homology matching or best-fit), consensus or germline sequences, or fragments of the framework sequences within each light or heavy chain variable region from several different human mAbs may be used. Alternatively the surface rodent residues may be replaced with the most common residues found in human mAbs (“resurfacing” or “veneering”).

The design of humanized immunoglobulins starting from a non-human antibody that has properties found to be critical in the present invention may be carried out as follows. As to the human framework region, a framework or variable region amino acid sequence of a CDR-providing non-human immunoglobulin is compared with corresponding sequences in a human immunoglobulin variable region sequence collection, and a sequence having a high percentage of identical amino acids is selected. When an amino acid falls under the following category, the framework amino acid of a human immunoglobulin to be used (acceptor immunoglobulin) is replaced by a framework amino acid from a CDR-providing non-human immunoglobulin (donor immunoglobulin):

(a) the amino acid in the human framework region of the acceptor immunoglobulin is unusual for human immunoglobulin at that position, whereas the corresponding amino acid in the donor immunoglobulin is typical for human immunoglobulin at that position;

(b) the position of the amino acid is immediately adjacent to one of the CDRs; or

(c) any side chain atom of a framework amino acid is within about 5-6 angstroms (center-to-center) of any atom of a CDR amino acid in a three dimensional immunoglobulin model [Queen, et al., Proc. Nat'l. Acad. Sci. (USA) 86:10029-10033 (1989); Co, et al., Proc. Nat'l. Acad. Sci. (USA) 88:2869 (1991)]. When each of the amino acid in the human framework region of the acceptor immunoglobulin and a corresponding amino acid in the donor immunoglobulin is unusual for human immunoglobulin at that position, such an amino acid is replaced by an amino acid typical for human immunoglobulin at that position.

Human antibodies may be readily obtained using known methods, such as, from human immune donors, from phage libraries, and from transgenic animals such as mice. Antibodies may be rescued from immune human donors using either EBV transformation of B-cells or by PCR cloning and phage display. Synthetic phage libraries may be created that use randomized combinations of synthetic human antibody V-regions. By selection on antigen, so called ‘fully human antibodies’ can be made, in which it is assumed that the V-regions are very human-like in nature. Transgenic mice can be created that have a repertoire of human immunoglobulin germline gene segments. These mice, when immunized with soluble Aβ, make human antibodies directed against soluble Aβ.

Preparation of high affinity humanized or human antibodies for use in the present invention may be carried out by methods well known in the art, including preparing monoclonal antibodies using well known techniques and screening for high affinity antibodies, or by first identifying a monoclonal antibody having reasonably high affinity and then improving the affinity using well known methods such as those described, for example, in: U.S. Pat. Nos. 5,976,562, 5,824514, 5,817,483, 5,814,476, 5,763,192, 5,723,323; WO97/29131; Thomas, et al., J. Biol. Chem. 277:2059-2064 (2002); Shreder, Methods 20:372-379 (2000); Boder, et al., Proc. Nat'l Acad. Sci. (USA) 97:10701-10705 (2000); Chen, et al., J. Mol. Biol. 293:865-881 (1999); Wu, et al., Proc. Nat'l. Acad. Sci. (USA) 95:6037-6042 (1998); Hoogenbloom, Trends Biotechnol. 15:62-70 (1997); Rader, et al., Curr. Opin. Biotechnol., 8:503-508 (1997); Crameri, et al., Nature Medicine 2:100-102 (1996); Crameri, et al., Nature Medicine 2:100-103 (1996); Schier, et al., J. Mol. Biol. 255:28-43 (1996); Yang, et al., J. Mol. Biol. 254:392-403 (1995); Yang, et al., J. Mol. Biol. 254:392-403 (1995); Yelton, et al., J Immunol. 0.155:1994-2004 (1995); Stemmer, Proc. Nat'l. Acad. Sci. (USA) 91:10747-10751 (1994); Stemmer, Nature 370:389-391 (1994); Huse, et al., Internat'l Rev. Immunol. 10:129-137 (1993); the portion of each of which having to do with preparation of high affinity antibodies is incorporated herein by reference.

The antibodies used in the present invention will most advantageously be expressed in recombinant hosts and purified using well known techniques [Page, et al., Bio/Technol. 9, 64-68 (1991); Carroll, et al., Mol. Immunol. 29, 821-827 (1992); Coloma, et al., J. Immunol. Meth. 152, 89-104 (1992); Bebbington, et al., Bio/Technol. 10, 169-175 (1992); Deyev, et al., FEBS Lett. 330, 111-113 (1993); Bender, et al., Hum. Antibodies Hybridomas 4, 74-79 (1993); Norderhaug, et al., J. Immunol. Meth. 204, 77-87 (1997); Poul, et al., Eur. J. Immunol. 25, 2005-2009 (1995), each of which is incorporated herein by reference].

A preferred antibody for use in the present invention is an antibody that binds to the same epitope on Aβ that 266 binds or any antibody that competitively inhibits the binding of 266 and human or mouse Aβ. The skilled reader will know how to determine, using well known methods, whether any particular antibody competitively inhibits the binding of 266 and human Aβ. For example, a competitive ELISA method could be used. Wells of a 96-well ELISA plate (e.g., Nunc-Immuno plate, Cat # 439454, NalgeNunc) are coated with Aβ peptide (1-40 is particularly convenient, but other lengths could be used also), optionally conjugated to a larger protein such as albumin. After washing the wells, they are blocked as appropriate, and then rinsed and dried appropriately. A mixture of biotinylated 266 antibody (e.g., biotinylated humanized 266, having as light chain the amino acid sequence of SEQ ID NO:11 and as heavy chain the amino acid sequence of SEQ ID NO:12) at 0.3 μg/ml final concentration, for example, and a competitor antibody (starting at 750 μg/ml final concentration and serial 3-fold dilutions) are added in a final volume of 100 μl per well. No-competitor and background controls are run. The ELISA plate is incubated at an appropriate temperature for an appropriate length of time, and then the wells are washed. After washing the wells, HRP-conjugated streptavidin (Cat # 21124, Pierce), or equivalent, is added to each well (e.g., 100 μl of 1 μg/ml). The plate is incubated at room temperature for an appropriate length of time, e.g., 30 min, and then is thoroughly washed. For color development, 100 μl/well of ABTS Peroxidase Substrate (Kirkegaard & Perry Laboratories), or equivalent, is added. Color development is stopped and absorbance is read (e.g., at 415 nm). The absorbances are plotted against the log of the competitor concentration, curves are fitted to the data points (e.g., using Prism or equivalent) and the IC50 determined using methods well known in the art. An antibody having an IC50 greater than 100-fold less than of that of 266 is considered to competitively inhibit the binding of 266 to Aβ. The affinity of an antibody for soluble Aβ can be determined using methods well known in the art or described herein.

Antibody 266 has the following amino acid sequences as CDRs:

light chain CDR1:

| | | | | | | | | | | | | | | | | |
|---|---|---|---|---|---|---|---|---|---|---|---|---|---|---|---|---|
| 1 | | | | 5 | | | | | 10 | | | | | 15 | | |
| Arg | Ser | Ser | Gln | Ser | Leu | Ile | Tyr | Ser | Asp | Gly | Asn | Ala | Tyr | Leu | His | (SEQ ID NO:1) |

light chain CDR2:

| | | | | | | | |
|---|---|---|---|---|---|---|---|
| 1 | | | | 5 | | | |
| Lys | Val | Ser | Asn | Arg | Phe | Ser | (SEQ ID NO:2) |

light chain CDR3:

| | | | | | | | | | |
|---|---|---|---|---|---|---|---|---|---|
| 1 | | | | 5 | | | | | |
| Ser | Gln | Ser | Thr | His | Val | Pro | Trp | Thr | (SEQ ID NO:3) |

light chain CDR1:

| | | | | | |
|---|---|---|---|---|---|
| 1 | | | | 5 | |
| Arg | Tyr | Ser | Met | Ser | (SEQ ID NO:4) |

heavy chain CDR2:

| | | | | | | | | | | | | | | | | | |
|---|---|---|---|---|---|---|---|---|---|---|---|---|---|---|---|---|---|
| 1 | | | | 5 | | | | | 10 | | | | | 15 | | | |
| Gln | Ile | Asn | Ser | Val | Gly | Asn | Ser | Thr | Tyr | Tyr | Pro | Asp | Thr | Val | Lys | Gly | (SEQ ID NO:5) |

and, heavy chain CDR3:

| | | | |
|---|---|---|---|
| 1 | | | |
| Gly | Asp | Tyr | (SEQ ID NO:6) |

In humanized versions of 266, human framework regions may optionally have substitutions of one to several residues from mouse 266 for the purpose of maintaining the strength or specificity of the binding of humanized antibody 266 [WO01/62801]. A preferred light chain variable region of a humanized 266 antibody has the following amino acid sequence:

| | | | | | | | | | | | | | | | | |
|---|---|---|---|---|---|---|---|---|---|---|---|---|---|---|---|---|
| 1 | | | | 5 | | | | | 10 | | | | | 15 | | |
| Asp | Xaa | Val | Met | Thr | Gln | Xaa | Pro | Leu | Ser | Leu | Pro | Val | Xaa | Xaa | Gly | (SEQ ID NO:7) |
| | | | 20 | | | | | 25 | | | | | 30 | | | |
| Gln | Pro | Ala | Ser | Ile | Ser | Cys | Arg | Ser | Ser | Gln | Ser | Leu | Xaa | Tyr | Ser | |
| | | 35 | | | | | 40 | | | | | 45 | | | | |
| Asp | Gly | Asn | Ala | Tyr | Leu | His | Trp | Phe | Leu | Gln | Lys | Pro | Gly | Gln | Ser | |
| | 50 | | | | | 55 | | | | | 60 | | | | | |
| Pro | Xaa | Leu | Leu | Ile | Tyr | Lys | Val | Ser | Asn | Arg | Phe | Ser | Gly | Val | Pro | |
| 65 | | | | | 70 | | | | | 75 | | | | | 80 | |
| Asp | Arg | Phe | Ser | Gly | Ser | Gly | Ser | Gly | Thr | Asp | Phe | Thr | Leu | Lys | Ile | |
| | | | | 85 | | | | | 90 | | | | | 95 | | |
| Ser | Arg | Val | Glu | Ala | Glu | Asp | Xaa | Gly | Val | Tyr | Tyr | Cys | Ser | Gln | Ser | |
| | | | 100 | | | | | 105 | | | | | 110 | | | |
| Thr | His | Val | Pro | Trp | Thr | Phe | Gly | Xaa | Gly | Thr | Xaa | Xaa | Glu | Ile | Lys | |
| Arg | | | | | | | | | | | | | | | | |

wherein:

Xaa at position 2 is Val or ile;

Xaa at position 7 is Ser or Thr;

Xaa at position 14 is Thr or Ser;

Xaa at position 15 is Leu or Pro;

Xaa at position 30 is Ile or Val;

Xaa at position 50 is Arg, Gln, or Lys;

Xaa at position 88 is Val or Leu;

Xaa at position 105 is Gln or Gly;

Xaa at position 108 is Lys or Arg; and

Xaa at position 109 is Val or Leu.

A preferred heavy chain variable region of a humanized 266 antibody has the following amino acid sequence:

| | | | | | | | | | | | | | | | | |
|---|---|---|---|---|---|---|---|---|---|---|---|---|---|---|---|---|
| 1 | | | | 5 | | | | | 10 | | | | | 15 | | |
| Xaa | Val | Gln | Leu | Val | Glu | Xaa | Gly | Gly | Gly | Leu | Val | Gln | Pro | Gly | Gly | (SEQ ID NO:8) |
| | | | 20 | | | | | 25 | | | | | 30 | | | |
| Ser | Leu | Arg | Leu | Ser | Cys | Ala | Ala | Ser | Gly | Phe | Thr | Phe | Ser | Arg | Tyr | |
| | | 35 | | | | | 40 | | | | | 45 | | | | |
| Ser | Met | Ser | Trp | Val | Arg | Gln | Ala | Pro | Gly | Lys | Gly | Leu | Xaa | Leu | Val | |
| | 50 | | | | | 55 | | | | | 60 | | | | | |
| Ala | Gln | Ile | Asn | Ser | Val | Gly | Asn | Ser | Thr | Tyr | Tyr | Pro | Asp | Xaa | Val | |
| 65 | | | | | 70 | | | | | 75 | | | | | 80 | |
| Lys | Gly | Arg | Phe | Thr | Ile | Ser | Arg | Asp | Asn | Xaa | Xaa | Asn | Thr | Leu | Tyr | |
| | | | | 85 | | | | | 90 | | | | | 95 | | |
| Leu | Gln | Met | Asn | Ser | Leu | Arg | Ala | Xaa | Asp | Thr | Ala | Val | Tyr | Tyr | Cys | |
| | | | 100 | | | | | 105 | | | | | 110 | | | |
| Ala | Ser | Gly | Asp | Tyr | Trp | Gly | Gln | Gly | Thr | Xaa | Val | Thr | Val | Ser | Ser | |

wherein:

Xaa at position 1 is Glu or Gln;

Xaa at position 7 is Ser or Leu;

Xaa at position 46 is Glu, Val, Asp, or Ser;

Xaa at position 63 is Thr or Ser;

Xaa at position 75 is Ala, Ser, Val, or Thr;

Xaa at position 76 is Lys or Arg;

Xaa at position 89 is Glu or Asp; and

Xaa at position 107 is Leu or Thr.

A particularly preferred light chain variable region of a humanized 266 antibody has the following amino acid sequence:

| | | | | | | | | | | | | | | | | |
|---|---|---|---|---|---|---|---|---|---|---|---|---|---|---|---|---|
| 1 | | | | 5 | | | | | 10 | | | | | 15 | | |
| Asp | Val | Val | Met | Thr | Gln | Ser | Pro | Leu | Ser | Leu | Pro | Val | Thr | Leu | Gly | (SEQ ID NO:9) |
| | | | 20 | | | | | 25 | | | | | 30 | | | |
| Gln | Pro | Ala | Ser | Ile | Ser | Cys | Arg | Ser | Ser | Gln | Ser | Leu | Ile | Tyr | Ser | |
| | | 35 | | | | | 40 | | | | | 45 | | | | |
| Asp | Gly | Asn | Ala | Tyr | Leu | His | Trp | Phe | Leu | Gln | Lys | Pro | Gly | Gln | Ser | |
| | 50 | | | | | 55 | | | | | 60 | | | | | |
| Pro | Arg | Leu | Leu | Ile | Tyr | Lys | Val | Ser | Asn | Arg | Phe | Ser | Gly | Val | Pro | |
| 65 | | | | | 70 | | | | | 75 | | | | | 80 | |
| Asp | Arg | Phe | Ser | Gly | Ser | Gly | Ser | Gly | Thr | Asp | Phe | Thr | Leu | Lys | Ile | |
| | | | | 85 | | | | | 90 | | | | | 95 | | |
| Ser | Arg | Val | Glu | Ala | Glu | Asp | Val | Gly | Val | Tyr | Tyr | Cys | Ser | Gln | Ser | |
| | | | 100 | | | | | 105 | | | | | 110 | | | |
| Thr | His | Val | Pro | Trp | Thr | Phe | Gly | Gln | Gly | Thr | Lys | Val | Glu | Ile | Lys | |
| Arg | | | | | | | | | | | | | | | | |

A particularly preferred heavy chain variable region of a humanized 266 antibody has the following amino acid sequence:

| | | | | | | | | | | | | | | | | |
|---|---|---|---|---|---|---|---|---|---|---|---|---|---|---|---|---|
| 1 | | | | 5 | | | | | 10 | | | | | 15 | | |
| Glu | Val | Gln | Leu | Val | Glu | Ser | Gly | Gly | Gly | Leu | Val | Gln | Pro | Gly | Gly | (SEQ ID NO:10) |
| | | | 20 | | | | | 25 | | | | | 30 | | | |
| Ser | Leu | Arg | Leu | Ser | Cys | Ala | Ala | Ser | Gly | Phe | Thr | Phe | Ser | Arg | Tyr | |
| | | 35 | | | | | 40 | | | | | 45 | | | | |
| Ser | Met | Ser | Trp | Val | Arg | Gln | Ala | Pro | Gly | Lys | Gly | Leu | Glu | Leu | Val | |
| | 50 | | | | | 55 | | | | | 60 | | | | | |
| Ala | Gln | Ile | Asn | Ser | Val | Gly | Asn | Ser | Thr | Tyr | Tyr | Pro | Asp | Thr | Val | |
| 65 | | | | | 70 | | | | | 75 | | | | | 80 | |
| Lys | Gly | Arg | Phe | Thr | Ile | Ser | Arg | Asp | Asn | Ala | Lys | Asn | Thr | Leu | Tyr | |
| | | | | 85 | | | | | 90 | | | | | 95 | | |
| Leu | Gln | Met | Asn | Ser | Leu | Arg | Ala | Glu | Asp | Thr | Ala | Val | Tyr | Tyr | Cys | |
| | | | 100 | | | | | 105 | | | | | 110 | | | |
| Ala | Ser | Gly | Asp | Tyr | Trp | Gly | Gln | Gly | Thr | Leu | Val | Thr | Val | Ser | Ser | |

A preferred light chain for a humanized 266 antibody has the amino acid sequence:

| | | | | | | | | | | | | | | | |
|---|---|---|---|---|---|---|---|---|---|---|---|---|---|---|---|
| 1 | | | | 5 | | | | | 10 | | | | | 15 | |
| Asp | Val | Val | Met | Thr | Gln | Ser | Pro | Leu | Ser | Leu | Pro | Val | Thr | Leu | (SEQ ID NO:11) |
| | | | | 20 | | | | | 25 | | | | | 30 | |
| Gly | Gln | Pro | Ala | Ser | Ile | Ser | Cys | Arg | Ser | Ser | Gln | Ser | Leu | Ile | |
| | | | | 35 | | | | | 40 | | | | | 45 | |
| Tyr | Ser | Asp | Gly | Asn | Ala | Tyr | Leu | His | Trp | Phe | Leu | Gln | Lys | Pro | |
| | | | | 50 | | | | | 55 | | | | | 60 | |
| Gly | Gln | Ser | Pro | Arg | Leu | Leu | Ile | Tyr | Lys | Val | Ser | Asn | Arg | Phe | |
| | | | | 65 | | | | | 70 | | | | | 75 | |
| Ser | Gly | Val | Pro | Asp | Arg | Phe | Ser | Gly | Ser | Gly | Ser | Gly | Thr | Asp | |
| | | | | 80 | | | | | 85 | | | | | 90 | |
| Phe | Thr | Leu | Lys | Ile | Ser | Arg | Val | Glu | Ala | Glu | Asp | Val | Gly | Val | |
| | | | | 95 | | | | | 100 | | | | | 105 | |
| Tyr | Tyr | Cys | Ser | Gln | Ser | Thr | His | Val | Pro | Trp | Thr | Phe | Gly | Gln | |
| | | | | 110 | | | | | 115 | | | | | 120 | |
| Gly | Thr | Lys | Val | Glu | Ile | Lys | Arg | Thr | Val | Ala | Ala | Pro | Ser | Val | |
| | | | | 125 | | | | | 130 | | | | | 135 | |
| Phe | Ile | Phe | Pro | Pro | Ser | Asp | Glu | Gln | Leu | Lys | Ser | Gly | Thr | Ala | |
| | | | | 140 | | | | | 145 | | | | | 150 | |
| Ser | Val | Val | Cys | Leu | Leu | Asn | Asn | Phe | Tyr | Pro | Arg | Glu | Ala | Lys | |
| | | | | 155 | | | | | 160 | | | | | 165 | |
| Val | Gln | Trp | Lys | Val | Asp | Asn | Ala | Leu | Gln | Ser | Gly | Asn | Ser | Gln | |
| | | | | 170 | | | | | 175 | | | | | 180 | |
| Glu | Ser | Val | Thr | Glu | Gln | Asp | Ser | Lys | Asp | Ser | Thr | Tyr | Ser | Leu | |
| | | | | 185 | | | | | 190 | | | | | 195 | |
| Ser | Ser | Thr | Leu | Thr | Leu | Ser | Lys | Ala | Asp | Tyr | Glu | Lys | His | Lys | |
| | | | | 200 | | | | | 205 | | | | | 210 | |
| Val | Tyr | Ala | Cys | Glu | Val | Thr | His | Gln | Gly | Leu | Ser | Ser | Pro | Val | |
| | | | | 215 | | | | | | | | | | | |
| Thr | Lys | Ser | Phe | Asn | Arg | Gly | Glu | Cys | | | | | | | |

A preferred heavy chain for a humanized 266 antibody has the amino acid sequence:

| | | | | | | | | | | | | | | | |
|---|---|---|---|---|---|---|---|---|---|---|---|---|---|---|---|
| 1 | | | | 5 | | | | | 10 | | | | | 15 | |
| Glu | Val | Gln | Leu | Val | Glu | Ser | Gly | Gly | Gly | Leu | Val | Gln | Pro | Gly | (SEQ ID NO:12) |
| | | | | 20 | | | | | 25 | | | | | 30 | |
| Gly | Ser | Leu | Arg | Leu | Ser | Cys | Ala | Ala | Ser | Gly | Phe | Thr | Phe | Ser | |
| | | | | 35 | | | | | 40 | | | | | 45 | |
| Arg | Tyr | Ser | Met | Ser | Trp | Val | Arg | Gln | Ala | Pro | Gly | Lys | Gly | Leu | |
| | | | | 50 | | | | | 55 | | | | | 60 | |
| Glu | Leu | Val | Ala | Gln | Ile | Asn | Ser | Val | Gly | Asn | Ser | Thr | Tyr | Tyr | |
| | | | | 65 | | | | | 70 | | | | | 75 | |
| Pro | Asp | Thr | Val | Lys | Gly | Arg | Phe | Thr | Ile | Ser | Arg | Asp | Asn | Ala | |
| | | | | 80 | | | | | 85 | | | | | 90 | |
| Lys | Asn | Thr | Leu | Tyr | Leu | Gln | Met | Asn | Ser | Leu | Arg | Ala | Glu | Asp | |
| | | | | 95 | | | | | 100 | | | | | 105 | |
| Thr | Ala | Val | Tyr | Tyr | Cys | Ala | Ser | Gly | Asp | Tyr | Trp | Gly | Gln | Gly | |
| | | | | 110 | | | | | 115 | | | | | 120 | |
| Thr | Leu | Val | Thr | Val | Ser | Ser | Ala | Ser | Thr | Lys | Gly | Pro | Ser | Val | |
| | | | | 125 | | | | | 130 | | | | | 135 | |
| Phe | Pro | Leu | Ala | Pro | Ser | Ser | Lys | Ser | Thr | Ser | Gly | Gly | Thr | Ala | |
| | | | | 140 | | | | | 145 | | | | | 150 | |
| Ala | Leu | Gly | Cys | Leu | Val | Lys | Asp | Tyr | Phe | Pro | Glu | Pro | Val | Thr | |
| | | | | 155 | | | | | 160 | | | | | 165 | |
| Val | Ser | Trp | Asn | Ser | Gly | Ala | Leu | Thr | Ser | Gly | Val | His | Thr | Phe | |
| | | | | 170 | | | | | 175 | | | | | 180 | |
| Pro | Ala | Val | Leu | Gln | Ser | Ser | Gly | Leu | Tyr | Ser | Leu | Ser | Ser | Val | |
| | | | | 185 | | | | | 190 | | | | | 195 | |
| Val | Thr | Val | Pro | Ser | Ser | Ser | Leu | Gly | Thr | Gln | Thr | Tyr | Ile | Cys | |
| | | | | 200 | | | | | 205 | | | | | 210 | |
| Asn | Val | Asn | His | Lys | Pro | Ser | Asn | Thr | Lys | Val | Asp | Lys | Lys | Val | |
| | | | | 215 | | | | | 220 | | | | | 225 | |
| Glu | Pro | Lys | Ser | Cys | Asp | Lys | Thr | His | Thr | Cys | Pro | Pro | Cys | Pro | |
| | | | | 230 | | | | | 235 | | | | | 240 | |
| Ala | Pro | Glu | Leu | Leu | Gly | Gly | Pro | Ser | Val | Phe | Leu | Phe | Pro | Pro | |
| | | | | 245 | | | | | 250 | | | | | 255 | |
| Lys | Pro | Lys | Asp | Thr | Leu | Met | Ile | Ser | Arg | Thr | Pro | Glu | Val | Thr | |
| | | | | 260 | | | | | 265 | | | | | 270 | |
| Cys | Val | Val | Val | Asp | Val | Ser | His | Glu | Asp | Pro | Glu | Val | Lys | Phe | |
| | | | | 275 | | | | | 280 | | | | | 285 | |
| Asn | Trp | Tyr | Val | Asp | Gly | Val | Glu | Val | His | Asn | Ala | Lys | Thr | Lys | |
| | | | | 290 | | | | | 295 | | | | | 300 | |
| Pro | Arg | Glu | Glu | Gln | Tyr | Asn | Ser | Thr | Tyr | Arg | Val | Val | Ser | Val | |
| | | | | 305 | | | | | 310 | | | | | 315 | |
| Leu | Thr | Val | Leu | His | Gln | Asp | Trp | Leu | Asn | Gly | Lys | Glu | Tyr | Lys | |
| | | | | 320 | | | | | 325 | | | | | 330 | |
| Cys | Lys | Val | Ser | Asn | Lys | Ala | Leu | Pro | Ala | Pro | Ile | Glu | Lys | Thr | |
| | | | | 335 | | | | | 340 | | | | | 345 | |
| Ile | Ser | Lys | Ala | Lys | Gly | Gln | Pro | Arg | Glu | Pro | Gln | Val | Tyr | Thr | |
| | | | | 350 | | | | | 355 | | | | | 360 | |
| Leu | Pro | Pro | Ser | Arg | Asp | Glu | Leu | Thr | Lys | Asn | Gln | Val | Ser | Leu | |
| | | | | 365 | | | | | 370 | | | | | 375 | |
| Thr | Cys | Leu | Val | Lys | Gly | Phe | Tyr | Pro | Ser | Asp | Ile | Ala | Val | Glu | |
| | | | | 380 | | | | | 385 | | | | | 390 | |
| Trp | Glu | Ser | Asn | Gly | Gln | Pro | Glu | Asn | Asn | Tyr | Lys | Thr | Thr | Pro | |
| | | | | 395 | | | | | 400 | | | | | 405 | |
| Pro | Val | Leu | Asp | Ser | Asp | Gly | Ser | Phe | Phe | Leu | Tyr | Ser | Lys | Leu | |
| | | | | 410 | | | | | 415 | | | | | 420 | |
| Thr | Val | Asp | Lys | Ser | Arg | Trp | Gln | Gln | Gly | Asn | Val | Phe | Ser | Cys | |
| | | | | 425 | | | | | 430 | | | | | 435 | |
| Ser | Val | Met | His | Glu | Ala | Leu | His | Asn | His | Tyr | Thr | Gln | Lys | Ser | |
| | | | | 440 | | | | | | | | | | | |
| Leu | Ser | Leu | Ser | Pro | Gly | Lys | | | | | | | | | |

A preferred antibody for use in the present invention is an analog of 266, in which an N-glycosylation site within CDR2 of the heavy chain (SEQ ID NO:5) is engineered so as not to be glycosylated. Such an analog has higher affinity for Aβ than 266 does, and comprises a light chain and a heavy chain, wherein the light chain comprises the three light chain complementarity determining regions (CDRs) from mouse monoclonal antibody 266 (SEQ ID NO:1-3), and wherein the heavy chain comprises heavy chain CDR1 and CDR3 from mouse monoclonal antibody 266 (SEQ ID NO:4 and 6, respectively), and a heavy chain CDR2 having the sequence given by SEQ ID NO:13:

| | | | | | | | | | | | | | | | | | |
|---|---|---|---|---|---|---|---|---|---|---|---|---|---|---|---|---|---|
| 1 | | | | 5 | | | | | 10 | | | | | 15 | | | |
| Gln | Ile | Asn | Ser | Val | Gly | Xaa | Xaa | Xaa | Tyr | Tyr | Pro | Asp | Thr | Val | Lys | Gly | (SEQ ID NO:13) |

wherein,

Xaa at position 7 is any amino acid, provided that if Xaa at position 8 is neither Asp nor Pro and Xaa at position 9 is Ser or Thr, then Xaa at position 7 is not Asn;

Xaa at position 8 is any amino acid, provided that if Xaa at position 7 is Asn and Xaa at position 9 is Ser or Thr, then Xaa at position 8 is Asp or Pro; and

Xaa at position 9 is any amino acid, provided that if Xaa at position 7 is Asn and Xaa at position 8 is neither Asp nor Pro, then Xaa at position 9 is neither Ser nor Thr.

By “any amino acid” is meant any naturally occurring amino acid. Preferred naturally-occurring amino acids are Ala, Cys, Asp, Glu, Phe, Gly, His, Ile, Lys, Leu, Met, Asn, Pro, Gln, Arg, Ser, Thr, Val, Trp, and Tyr.

A preferred group of antibodies are those having as light chain CDR1-CDR3 the sequences SEQ ID NO:1-3, respectively, as heavy chain CDR1 and CDR3 the sequences SEQ ID NO:4 and 6, respectively, and wherein the sequence of heavy chain CDR2 is SEQ ID NO:13, wherein:

Xaa at position 7 of SEQ ID NO:13 is selected from the group consisting of Ala, Cys, Asp, Glu, Phe, Gly, His, Ile, Lys, Leu, Met, Asn, Pro, Gln, Arg, Ser, Thr, Val, Trp, and Tyr, provided that if Xaa at position 8 is neither Asp nor Pro and Xaa at position 9 is Ser or Thr, then Xaa at position 7 is not Asn;

Xaa at position 8 of SEQ ID NO:13 is selected from the group consisting of Ala, Cys, Asp, Glu, Phe, Gly, His, Ile, Lys, Leu, Met, Asn, Pro, Gln, Arg, Ser, Thr, Val, Trp, and Tyr, provided that if Xaa at position 7 is Asn and Xaa at position 9 is Ser or Thr, then Xaa at position 8 is Asp or Pro; and

Xaa at position 9 of SEQ ID NO:13 is selected from the group consisting of Ala, Cys, Asp, Glu, Phe, Gly, His, Ble, Lys, Leu, Met, Asn, Pro, Gln, Arg, Ser, Thr, Val, Trp, and Tyr, provided that if Xaa at position 7 is Asn and Xaa at position 8 is neither Asp nor Pro, then Xaa at position 9 is neither Ser nor Thr.

Another description of the preferred group is: antibodies or fragments thereof having as light chain CDR1-CDR3 the sequences SEQ ID NO:1-3, respectively, as heavy chain CDR1 and CDR3 the sequences SEQ ID NO:4 and 6, respectively, and wherein the sequence of heavy chain CDR2 is selected from the group consisting of:

1) SEQ ID NO:14

| | |
|---|---|
| 1 5 10 15 | |
| Gln Ile Asn Ser Val Gly Xaa Xaa Xaa Tyr Tyr Pro Asp Thr Val Lys Gly | (SEQ ID NO:14) |

wherein:

Xaa at position 7 of SEQ ID NO:14 is selected from the group consisting of Ala, Cys, Asp, Glu, Phe, Gly, His, Ile, Lys, Leu, Met, Pro, Gln, Arg, Ser, Thr, Val, Trp, and Tyr;

Xaa at position 8 of SEQ ID NO:14 is selected from the group consisting of Ala, Cys, Asp, Glu, Phe, Gly, His, Ble, Lys, Leu, Met, Asn, Pro, Gln, Arg, Ser, Thr, Val, Trp, and Tyr; and

Xaa at position 9 of SEQ ID NO:14 is selected from the group consisting of Ala, Cys, Asp, Glu, Phe, Gly, His, Ile, Lys, Leu, Met, Asn, Pro, Gln, Arg, Ser, Thr, Val, Trp, and Tyr;

2) SEQ ID NO:15

| | |
|---|---|
| 1 5 10 15 | |
| Gln Ile Asn Ser Val Gly Xaa Xaa Xaa Tyr Tyr Pro Asp Thr Val Lys Gly | (SEQ ID NO:15) |

wherein:

Xaa at position 7 of SEQ ID NO:15 is Asn;

Xaa at position 8 of SEQ ID NO:15 is selected from the group consisting of Ala, Cys, Asp, Glu, Phe, Gly, His, Ile, Lys, Leu, Met, Asn, Pro, Gln, Arg, Ser, Thr, Val, Trp, and Tyr; and

Xaa at position 9 of, SEQ ID NO:15 is selected from the group consisting of Ala, Cys, Asp, Glu, Phe, Gly, His, Ile, Lys, Leu, Met, Asn, Pro, Gln, Arg, Val, Trp, and Tyr; and

3) SEQ ID NO:16

| | |
|---|---|
| 1 5 10 15 | |
| Gln Ile Asn Ser Val Gly Xaa Xaa Xaa Tyr Tyr Pro Asp Thr Val Lys Gly | (SEQ ID NO:16) |

wherein:

Xaa at position 7 of SEQ ID NO:16 is Asn;

Xaa at position 8 of SEQ ID NO:16 is selected from the group consisting of Asp and Pro; and

Xaa at position 9 of SEQ ID NO:16 is selected from the group consisting of Ser and Thr.

Preferred sequences for CDR2 of the heavy chain include those in which only a single amino acid is changed, those in which only two amino acids are changed, or all three are changed. It is preferred to replace Asn at position 7, or to replace Thr at position 9, or to replace both. Conservative substitutions at one, two, or all three positions are preferred. The most preferred species are those in which Asn at position 7 is replaced with Ser or Thr.

Preferred deglycosylated 266 antibodies for use in the present invention are those in which in CDR2 of the heavy chain (i.e., within SEQ ID NO:13, as described above):

Xaa at position 7 is selected from the group consisting of Ala, Gly, His, Asn, Gln, Ser, and Thr, provided that if Xaa at position 9 is Ser or Thr, then Xaa at position 7 is not Asn;

Xaa at position 8 is selected from the group consisting of Ala, Gly, His, Asn, Gln, Ser, and Thr; and

Xaa at position 9 is selected from the group consisting of Ala, Gly, His, Asn, Gin, Ser, and Thr, provided that if Xaa at position 7 is Asn, then Xaa at position 9 is neither Ser nor Thr.

An alternate description of preferred declycogsylated 266 antibodies is: antibodies or fragments thereof having as light chain CDR1-CDR3 the sequences SEQ ID NO:1-3, respectively, as heavy chain CDR1 and CDR3 the sequences SEQ ID NO:4 and 6, respectively, and wherein the sequence of heavy chain CDR2 is selected from the group consisting of:

1) SEQ ID NO:17

| | |
|---|---|
| 1 5 10 15 | |
| Gln Ile Asn Ser Val Gly Xaa Xaa Xaa Tyr Tyr Pro Asp Thr Val Lys Gly | (SEQ ID NO:17) |

wherein:

Xaa at position 7 of SEQ ID NO:17 is selected from the group consisting of Ala, Gly, His, Gln, Ser, and Thr;

Xaa at position 8 of SEQ ID NO:17 is selected from the group consisting of Ala, Gly, His, Asn, Gln, Ser, and Thr; and

Xaa at position 9 of SEQ ID NO:17 is selected from the group consisting of Ala, Gly, His, Asn, Gin, Ser, and Thr; and

2) SEQ ID NO:18

| | |
|---|---|
| 1 5 10 15 | |
| Gln Ile Asn Ser Val Gly Xaa Xaa Xaa Tyr Tyr Pro Asp Thr Val Lys Gly | (SEQ ID NO:18) |

wherein:

Xaa at position 7 of SEQ ID NO:18 is Asn;

Xaa at position 8 of SEQ ID NO:18 is selected from the group consisting of Ala, Gly, His, Asn, Gln, Ser, and Thr; and

Xaa at position 9 of SEQ ID NO:18 is selected from the group consisting of Ala, Gly, His, Asn, and Gln.

A preferred humanized antibody for use in the present invention has the light chain variable region of SEQ ID NO:7 and a heavy chain variable region of SEQ ID NO:19:

| | | | | | | | | | | | | | | | |
|---|---|---|---|---|---|---|---|---|---|---|---|---|---|---|---|
| 1 | | | | 5 | | | | | 10 | | | | | 15 | |
| Xaa | Val | Gln | Leu | Val | Glu | Xaa | Gly | Gly | Gly | Leu | Val | Gln | Pro | Gly | (SEQ ID NO:19) |
| | | | | 20 | | | | | 25 | | | | | 30 | |
| Gly | Ser | Leu | Arg | Leu | Ser | Cys | Ala | Ala | Ser | Gly | Phe | Thr | Phe | Ser | |
| | | | | 35 | | | | | 40 | | | | | 45 | |
| Arg | Tyr | Ser | Met | Ser | Trp | Val | Arg | Gln | Ala | Pro | Gly | Lys | Gly | Leu | |
| | | | | 50 | | | | | 55 | | | | | 60 | |
| Xaa | Leu | Val | Ala | Gln | Ile | Asn | Ser | Val | Gly | Xaa | Xaa | Xaa | Tyr | Tyr | |
| | | | | 65 | | | | | 70 | | | | | 75 | |
| Pro | Asp | Xaa | Val | Lys | Gly | Arg | Phe | Thr | Ile | Ser | Arg | Asp | Asn | Xaa | |
| | | | | 80 | | | | | 85 | | | | | 90 | |
| Xaa | Asn | Thr | Leu | Tyr | Leu | Gln | Met | Asn | Ser | Leu | Arg | Ala | Xaa | Asp | |
| | | | | 95 | | | | | 100 | | | | | 105 | |
| Thr | Ala | Val | Tyr | Tyr | Cys | Ala | Ser | Gly | Asp | Tyr | Trp | Gly | Gln | Gly | |
| | | | | 110 | | | | | | | | | | | |
| Thr | Xaa | Val | Thr | Val | Ser | Ser | | | | | | | | | |

wherein:

Xaa at position 1 is Glu or Gln;

Xaa at position 7 is Ser or Leu;

Xaa at position 46 is Glu, Val, Asp, or Ser;

Xaa at position 56 is any amino acid, provided that if Xaa at position 57 is neither Asp nor Pro and Xaa at position 59 is Ser or Thr, then Xaa at position 56 is not Asn;

Xaa at position 57 is any amino acid, provided that if Xaa at position 56 is Asn and Xaa at position 58 is Ser or Thr, then Xaa at position 57 is Asp or Pro; and

Xaa at position 58 is any amino acid, provided that if Xaa at position 56 is Asn and Xaa at position 57 is neither Asp nor Pro, then Xaa at position 58 is neither Ser nor Thr

Xaa at position 63 is Thr or Ser;

Xaa at position 75 is Ala, Ser, Val, or Thr;

Xaa at position 76 is Lys or Arg;

Xaa at position 89 is Glu or Asp; and

Xaa at position 107 is Leu or Thr.

A preferred humanized antibody for use in the present invention has the light chain variable region of SEQ ID NO:9 and a heavy chain variable region of SEQ ID NO:20:

| | | | | | | | | | | | | | | | |
|---|---|---|---|---|---|---|---|---|---|---|---|---|---|---|---|
| 1 | | | | 5 | | | | | 10 | | | | | 15 | |
| Glu | Val | Gln | Leu | Val | Glu | Ser | Gly | Gly | Gly | Leu | Val | Gln | Pro | Gly | (SEQ ID NO:20) |
| | | | | 20 | | | | | 25 | | | | | 30 | |
| Gly | Ser | Leu | Arg | Leu | Ser | Cys | Ala | Ala | Ser | Gly | Phe | Thr | Phe | Ser | |
| | | | | 35 | | | | | 40 | | | | | 45 | |
| Arg | Tyr | Ser | Met | Ser | Trp | Val | Arg | Gln | Ala | Pro | Gly | Lys | Gly | Leu | |
| | | | | 50 | | | | | 55 | | | | | 60 | |
| Glu | Leu | Val | Ala | Gln | Ile | Asn | Ser | Val | Gly | Xaa | Xaa | Xaa | Tyr | Tyr | |
| | | | | 65 | | | | | 70 | | | | | 75 | |
| Pro | Asp | Thr | Val | Lys | Gly | Arg | Phe | Thr | Ile | Ser | Arg | Asp | Asn | Ala | |
| | | | | 80 | | | | | 85 | | | | | 90 | |
| Lys | Asn | Thr | Leu | Tyr | Leu | Gln | Met | Asn | Ser | Leu | Arg | Ala | Glu | Asp | |
| | | | | 95 | | | | | 100 | | | | | 105 | |
| Thr | Ala | Val | Tyr | Tyr | Cys | Ala | Ser | Gly | Asp | Tyr | Trp | Gly | Gln | Gly | |
| | | | | 110 | | | | | | | | | | | |
| Thr | Leu | Val | Thr | Val | Ser | Ser | | | | | | | | | |

wherein:

Xaa at position 56 is any amino acid, provided that if Xaa at position 57 is neither Asp nor Pro and Xaa at position 59 is Ser or Thr, then Xaa at position 56 is not Asn;

Xaa at position 57 is any amino acid, provided that if Xaa at position 56 is Asn and Xaa at position 58 is Ser or Thr, then Xaa at position 57 is Asp or Pro; and

Xaa at position 58 is any amino acid, provided that if Xaa at position 56 is Asn and Xaa at position 57 is neither Asp nor Pro, then Xaa at position 58 is neither Ser nor Thr.

A preferred humanized antibody for use in the present invention has the light chain variable region of SEQ ID NO:11 and a heavy chain given by SEQ ID NO:21:

(SEQ ID NO:21)

| | | | | | | | | | | | | | | |
|---|---|---|---|---|---|---|---|---|---|---|---|---|---|---|
| 1 | | | | 5 | | | | | 10 | | | | | 15 |
| Glu | Val | Gln | Leu | Val | Glu | Ser | Gly | Gly | Gly | Leu | Val | Gln | Pro | Gly |
| | | | | 20 | | | | | 25 | | | | | 30 |
| Gly | Ser | Leu | Arg | Leu | Ser | Cys | Ala | Ala | Ser | Gly | Phe | Thr | Phe | Ser |
| | | | | 35 | | | | | 40 | | | | | 45 |
| Arg | Tyr | Ser | Met | Ser | Trp | Val | Arg | Gln | Ala | Pro | Gly | Lys | Gly | Leu |
| | | | | 50 | | | | | 55 | | | | | 60 |
| Glu | Leu | Val | Ala | Gln | Ile | Asn | Ser | Val | Gly | Xaa | Xaa | Xaa | Tyr | Tyr |
| | | | | 65 | | | | | 70 | | | | | 75 |
| Pro | Asp | Thr | Val | Lys | Gly | Arg | Phe | Thr | Ile | Ser | Arg | Asp | Asn | Ala |
| | | | | 80 | | | | | 85 | | | | | 90 |
| Lys | Asn | Thr | Leu | Tyr | Leu | Gln | Met | Asn | Ser | Leu | Arg | Ala | Glu | Asp |
| | | | | 95 | | | | | 100 | | | | | 105 |
| Thr | Ala | Val | Tyr | Tyr | Cys | Ala | Ser | Gly | Asp | Tyr | Trp | Gly | Gln | Gly |
| | | | | 110 | | | | | 115 | | | | | 120 |
| Thr | Leu | Val | Thr | Val | Ser | Ser | Ala | Ser | Thr | Lys | Gly | Pro | Ser | Val |
| | | | | 125 | | | | | 130 | | | | | 135 |
| Phe | Pro | Leu | Ala | Pro | Ser | Ser | Lys | Ser | Thr | Ser | Gly | Gly | Thr | Ala |
| | | | | 140 | | | | | 145 | | | | | 150 |
| Ala | Leu | Gly | Cys | Leu | Val | Lys | Asp | Tyr | Phe | Pro | Glu | Pro | Val | Thr |
| | | | | 155 | | | | | 160 | | | | | 165 |
| Val | Ser | Trp | Asn | Ser | Gly | Ala | Leu | Thr | Ser | Gly | Val | His | Thr | Phe |
| | | | | 170 | | | | | 175 | | | | | 180 |
| Pro | Ala | Val | Leu | Gln | Set | Ser | Gly | Leu | Tyr | Ser | Leu | Ser | Ser | Val |
| | | | | 185 | | | | | 190 | | | | | 195 |
| Val | Thr | Val | Pro | Ser | Ser | Ser | Leu | Gly | Thr | Gln | Thr | Tyr | Ile | Cys |
| | | | | 200 | | | | | 205 | | | | | 210 |
| Asn | Val | Asn | His | Lys | Pro | Ser | Asn | Thr | Lys | Val | Asp | Lys | Lys | Val |
| | | | | 215 | | | | | 220 | | | | | 225 |
| Glu | Pro | Lys | Ser | Cys | Asp | Lys | Thr | His | Thr | Cys | Pro | Pro | Cys | Pro |
| | | | | 230 | | | | | 235 | | | | | 240 |
| Ala | Pro | Glu | Leu | Leu | Gly | Gly | Pro | Ser | Val | Phe | Leu | Phe | Pro | Pro |
| | | | | 245 | | | | | 250 | | | | | 255 |
| Lys | Pro | Lys | Asp | Thr | Leu | Met | Ile | Ser | Arg | Thr | Pro | Glu | Val | Thr |
| | | | | 260 | | | | | 265 | | | | | 270 |
| Cys | Val | Val | Val | Asp | Val | Ser | His | Glu | Asp | Pro | Glu | Val | Lys | Phe |
| | | | | 275 | | | | | 280 | | | | | 285 |
| Asn | Trp | Tyr | Val | Asp | Gly | Val | Glu | Val | His | Asn | Ala | Lys | Thr | Lys |
| | | | | 290 | | | | | 295 | | | | | 300 |
| Pro | Arg | Glu | Glu | Gln | Tyr | Asn | Ser | Thr | Tyr | Arg | Val | Val | Ser | Val |
| | | | | 305 | | | | | 310 | | | | | 315 |
| Leu | Thr | Val | Leu | His | Gln | Asp | Trp | Leu | Asn | Gly | Lys | Glu | Tyr | Lys |
| | | | | 320 | | | | | 325 | | | | | 330 |
| Cys | Lys | Val | Ser | Asn | Lys | Ala | Leu | Pro | Ala | Pro | Ile | Glu | Lys | Thr |
| | | | | 335 | | | | | 340 | | | | | 345 |
| Ile | Ser | Lys | Ala | Lys | Gly | Gln | Pro | Arg | Glu | Pro | Gln | Val | Tyr | Thr |
| | | | | 350 | | | | | 355 | | | | | 360 |
| Leu | Pro | Pro | Ser | Arg | Asp | Glu | Leu | Thr | Lys | Asn | Gln | Val | Ser | Leu |
| | | | | 365 | | | | | 370 | | | | | 375 |
| Thr | Cys | Leu | Val | Lys | Gly | Phe | Tyr | Pro | Ser | Asp | Ile | Ala | Val | Glu |
| | | | | 380 | | | | | 385 | | | | | 390 |
| Trp | Glu | Ser | Asn | Gly | Gln | Pro | Glu | Asn | Asn | Tyr | Lys | Thr | Thr | Pro |
| | | | | 395 | | | | | 400 | | | | | 405 |
| Pro | Val | Leu | Asp | Ser | Asp | Gly | Ser | Phe | Phe | Leu | Tyr | Ser | Lys | Leu |
| | | | | 410 | | | | | 415 | | | | | 420 |
| Thr | Val | Asp | Lys | Ser | Arg | Trp | Gln | Gln | Gly | Asn | Val | Phe | Ser | Cys |
| | | | | 425 | | | | | 430 | | | | | 435 |
| Ser | Val | Met | His | Glu | Ala | Leu | His | Asn | His | Tyr | Thr | Gln | Lye | Ser |
| | | | | 440 | | | | | | | | | | |
| Leu | Ser | Leu | Ser | Pro | Gly | Lys | | | | | | | | |

wherein:

Xaa at position 56 is any amino acid, provided that if Xaa at position 57 is neither Asp nor Pro and Xaa at position 59 is Ser or Thr, then Xaa at position 56 is not Asn;

Xaa at position 57 is any amino acid, provided that if Xaa at position 56 is Asn and Xaa at position 58 is Ser or Thr, then Xaa at position 57 is Asp or Pro; and

Xaa at position 58 is any amino acid, provided that if Xaa at position 56 is Asn and Xaa at position 57 is neither Asp nor Pro, then Xaa at position 58 is neither Ser nor Thr.

Preferred deglycosylated 266 antibodies having the heavy variable region according to SEQ ID NO:19, SEQ ID NO:20, and SEQ ID NO:21 are those wherein:

Xaa at position 56 is selected from the group consisting of Ala, Gly, His, Asn, Gln, Ser, and Thr, provided that if Xaa at position 58 is Ser or Thr, then Xaa at position 56 is not Asn;

Xaa at position 57 is selected from the group consisting of Ala, Gly, His, Asn, Gln, Ser, and Thr; and

Xaa at position 58 is selected from the group consisting of Ala, Gly, His, Asn, Gin, Ser, and Thr, provided that if Xaa at position 56 is Asn, then Xaa at position 58 is neither Ser nor Thr.

Preferred sequences for CDR2 (positions 56, 57, and 58) of the heavy chain SEQ ID NO:19, SEQ ID NO:20, and SEQ ID NO:21 include those in which only a single amino acid is changed, those in which only two amino acids are changed, or all three are changed. It is preferred to replace Asn at position 56. It is preferred to replace Thr at position 58 with an amino acid other than Ser. It is preferred to not destroy the N-glycosylation site in the CDR2 of the 266 heavy chain by replacing Ser at position 57 with Pro or Asp. Conservative substitutions at one, two, or all three positions are preferred. The most preferred species are those in which Asn at position 56 is replaced with Ser or Thr. Particularly preferred antibodies are those in which Ser or Thr is at position 56, Ser is at position 57, and Thr is at position 58 of SEQ ID NO:19, SEQ ID NO:20, or SEQ ID NO:21.

The most preferred species are antibodies comprising a light chain of SEQ ID NO:11 and a heavy chain of SEQ ID NO:21, wherein in SEQ ID NO:21, Xaa at position 56 is Ser, Xaa at position 57 is Ser, and Xaa at position 58 is Thr (“N56S”), or wherein in SEQ ID NO:21, Xaa at position 56 is Thr, Xaa at position 57 is Ser, and Xaa at position 58 is Thr (“N56T”).

The antibodies (including immunologically reactive fragments) are administered to a subject as identified above using standard parenteral, peripheral administration techniques, by intravenous, intraperitoneal, subcutaneous, pulmonary, transdermal, intramuscular, intranasal, buccal, sublingual, or suppository administration. The preferred routes of administration are intravenous, subcutaneous, and intraperitoneal.

The preparation of an acceptable pharmaceutical preparation of the antibodies used in the present invention, including its strength, excipients, pH, isotonicity, presentation, dosage form, and the like, is well known to the skilled person. Pharmaceutical compositions for use in the present invention should be appropriate for the selected mode of administration, and pharmaceutically acceptable excipients such as, buffers, surfactants, preservatives, solubilizing agents, isotonicity agents, stabilizing agents and the like are used as appropriate. Remington's Pharmaceutical Sciences, Mack Publishing Co., Easton Pa., latest edition, incorporated herein by reference, provides a compendium of formulation techniques as are generally known to practitioners. Pharmaceutical preparations for use in the present invention should be sterile or at least nearly so, and if necessary preserved or rendered bacteriostatic.

The following examples are intended to illustrate, not limit, the invention. The examples describe experiments conducted in murine systems, thus the use of murine monoclonal antibodies is satisfactory. However, in the treatment methods of the invention intended for human use, humanized or fully human antibodies are preferred.

### EXAMPLES

### Example 1

### Rapid Improvement in Cognition after Administration of Anti-Aβ Antibody 266

APPV717F transgenic mice (PDAPP mice, eleven month old) were used [Games et al., Nature 373:523-527 (1995)]. The mice were handled daily 5 days before the behavioral testing. All animals had free access to food and water. They were housed at a room temperature of 23±1° C. and with a light-dark cycle of 12:12 h with lights on at 6:00 a.m. Behavioral experiments were conducted during the light period, between 8:00 a.m. and 2 p.m.

The object recognition task is based on the spontaneous tendency of rodents to explore a novel object more often than a familiar one [Ennaceur et Delacour, Behavioral Brain Research. 31:47-59 (1988); Dodart et al., Neuroreport 8:1173-1178 (1997)]. This task was performed in a black PlexiglasTM open field (50×50×40 cm). The objects to be discriminated were a marble (1.5 cm diameter) and a plastic dice (1.8 cm edge). After each trial, the objects were handled with disposable gloves and immersed in alcohol to eliminate olfactory cues. Before experiments, several male mice were placed in the open field in order to condition the testing environment. On the first day of testing, mice were submitted to a familiarization session by placing them in the empty open field for 30 min and the distance traveled (cm) was recorded by at 5-minute intervals using a computer-assisted video tracking system (San Diego Instrument, CA). On the following day, mice were submitted to two 10 min trials with a 3 hour inter-trial delay. During trial 1, mice were allowed to explore the open field in the presence of object A (marble or dice). The distance traveled (cm) and the time spent exploring the object (nose pointing toward the object at a distance≦1 cm) were recorded with the video tracking system and by hand, respectively. For trial 2, mice were allowed to explore the open field in the presence of two objects: the familiar object (“object A”) and a novel object (“object B”) (e.g., a marble and a die).

A recognition index calculated for each animal was expressed by the ratio (t_{B}×100)/(t_{A}+t_{B}) where t_{A} and t_{B} are the time spent on object A and object B respectively. An observer blind to the treatment status of the animals recorded the object exploration time.

Murine anti-Aβ antibody 266 [“m266”, Seubert, et al., Nature 359:325-327 (1992)] and a purified mouse IgG1 isotype control (Pharmingen) were diluted from stock solutions in PBS prior to administration. Animals were injected (500 μL, i.p.) with m266 (n=8, 1 mg/mL) control IgG solution (n=8, 1 mg/mL) or PBS (n=7) 3 hours before the familiarization session, which corresponds to 24 hours before trial 1. An additional control group of wild-type mice (WT) animals was tested in parallel in the object recognition task (n=6).

After completion of trial 2, blood and CSF fluids were sampled, and brains were processed using a 3-step extraction procedure. The first step consisted of homogenizing samples in cold PBS and complex of proteinase inhibitors (Complete™, Boehringer-Mannheim, Ind.) followed by centrifugation at 10,000 rpm for 10 min at 4° C., the supernatant was considered as the PBS “soluble” pool. The second step consisted of re-suspension of the pellet in RIPA (50 mM Tris, 150 mM NaCl, 0.5% DOC, 1% NP40, 0.1% SDS and Complete™, pH 8.0) followed by centrifugation at 10,000 rpm for 10 min at 4° C., the supernatant was designated the “detergent soluble” pool. Finally, the third step consisted of re-suspension of the pellet in 5M Guanidine-HCl, rocking the tubes for 2 hours at room temperature, followed by centrifugation at 10,000 rpm for 10 min at 4° C. This step produced the “insoluble” pool. Aβ40 and Aβ42 were quantified in each pool using an ELISA [Bales, et al., Proc. Natl. Acad. Sci. USA. 96:15233-15238 (1999)].

Briefly, for the ELISA, the monoclonal antibodies 2G3 and 21F12 were used to capture Aβ peptides terminating at residues 40 and 42 respectively [Johnson-Wood, et al., 1997]. Biotinylated 3D6, which recognizes the Aβ 1-5 region, was used as the reporter antibody.

Proteins of plasma and CSF samples were separated by electrophoresis under non-denaturing conditions utilizing a 4-20% TBE gel (Criterion gel, Bio Rad, Calif.) and transferred in CAPS buffer (CAPS 10 mM, 0.01% SDS, 1% Methanol, pH 11) onto PVDF membrane. After a 1-hour block in SuperBlock blocking buffer (Pierce, Ill.), the membrane was probed with biotinylated 3D6 (0.045 mg/ml), thereafter reacted with StreptAvidin (1:200000) and visualized utilizing SuperSignal West Femto (Pierce, Ill.).

To compare behavioral data as well as the Aβ levels between groups, one-way or two-way analyses of variance (ANOVA) and correlation analyses were performed using the Statview5 software (SAS Institute Inc., NC).

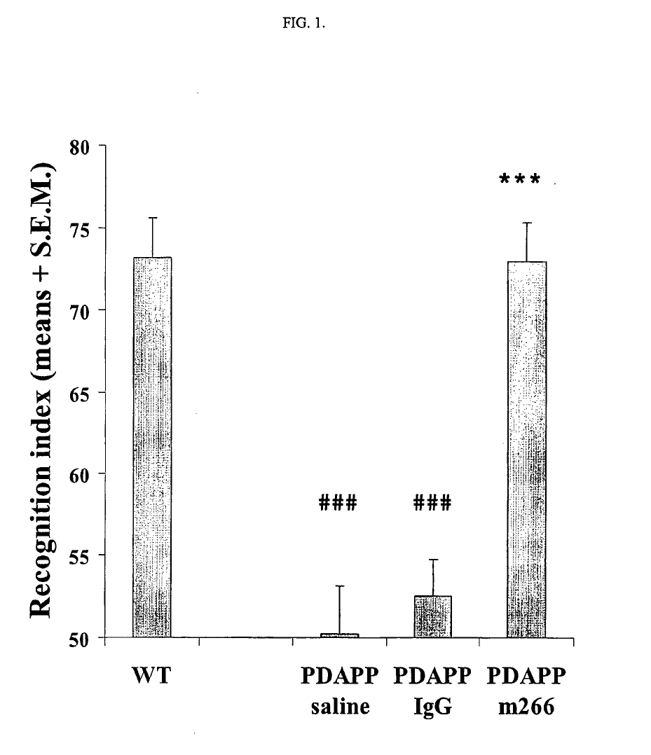

As shown in FIG. 1, the object recognition index differed significantly between treatment groups (group effect: F_{3,25}=25.085, p<0.0001). Performance of m266-treated tg mice was significantly higher than performance of saline-treated tg mice and control IgG-treated tg mice (p<0.0001) and comparable to WT mice. The object recognition index did not differ between saline- and control IgG-treated tg mice. Moreover, a within-group t-test analysis confirmed that m266-(t-value=9.526, p<0.0001) treated tg mice and WT mice (t-value=9.581, p<0.0002) performed above chance levels (50%) whereas saline- and control IgG-treated tg mice did not (t-values=0.081 and 1.157 respectively, NS). A group effect analysis also confirmed that there were no differences between groups in total object exploration time during trial 1 (F_{3,25}=0.555, NS) or trial 2 (F_{3,25}=0.679, NS). The distance traveled also did not differ between groups during the familiarization session (group×block interaction: F_{15,125}=1.455, NS; group effect: F_{3,25}=1.200, NS), during trial 1 (group effect: F_{3,25}=1.326, NS) or during trial 2 (group effect: F_{3,23}=1.334, NS).

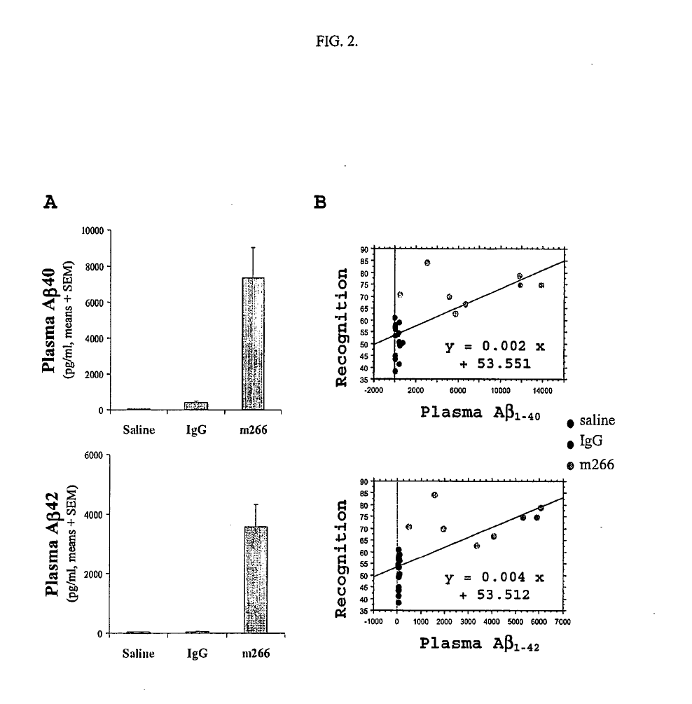

Following administration of m266, as detected by ELISA (FIG. 2a), the levels of both Aβ40 and Aβ42 peptides were significantly increased in plasma samples when compared to saline- and IgG-treated mice (p<0.0001). The increase for Aβ40, when compared to saline-treated mice was approximately 150-fold and the increase for Aβ42, was approximately 70-fold. Neither Aβ40 nor Aβ42 levels differed between saline- and control IgG-treated mice. Remarkably, the plasma levels of both Aβ peptides following m266 treatment were highly correlated with object recognition memory performance (r=0.774, p<0.0001 for Aβ40; r=0.781, p<0.0001 for Aβ42) (FIG. 2b).

In CSF samples, levels of Aβ40 significantly differed between groups (F_{2,19}=4.798, p<0.05), m266-treated animals showing increased levels of this peptide compared to saline- or control IgG-treated animals (p<0.05, levels of Aβ40 in ng per ml of protein: saline, 7.79±1.73; control IgG, 8.72±2.9; m266, 16.57±3.25). No difference in CSF levels of Aβ42 was observed between groups (F_{2,19}=3.006, NS).

In the cerebral cortex, a significant difference between groups was observed only for Aβ40 in the PBS-soluble pool (F_{2,20}=7.785, p<0.01); m266-treated mice showing increased levels of soluble Aβ peptide. No significant differences in levels of Aβ were found between groups in the hippocampal extracts (data not shown). Interestingly, object recognition performance was also significantly correlated with levels of Aβ40 in CSF and in the cortical PBS-soluble pool (r=0.491, p<0.05 and r=0.605, p<0.01).

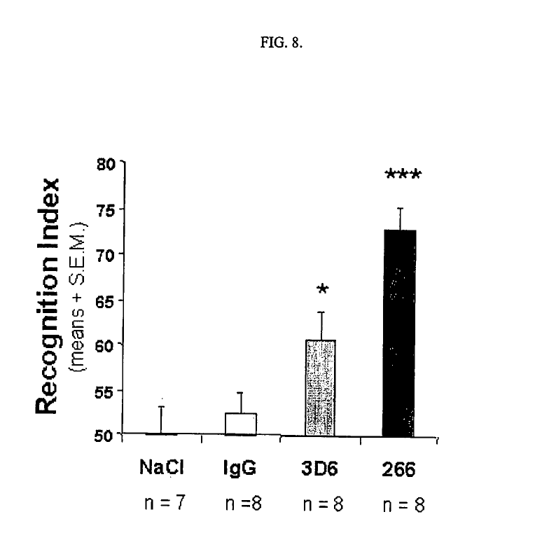

Administration of 360 μg of 3D6 per animal 24 hours prior to the habituation session in 11-12 month old APPV717F tg mice improved OR performance in only 2 of 8 mice tested (FIG. 8) (p<0.05). This improvement was much less significant than that obtained using antibody 266 that has a significantly greater affinity for soluble Aβ than 3D6, and a significantly greater ability to cause flux of Aβ from brain than does 3D6.

These results demonstrate that administration of an antibody having a very high affinity for soluble Aβ (but a very low affinity for insoluble Aβ) rapidly and efficiently reverses object recognition memory impairments in APPV717F transgenic nice. This rapid improvement in cognitive function is accompanied by significant and marked increases in plasma, CSF and cortical soluble levels of Aβ, but not by any measurable changes in the brain insoluble pool of Aβ.

Example 2

### Rapid Effect of Administration of Anti-Aβ Antibodies on Cognition Correlated with Affinity for Soluble Aβ

The anti-Aβ murine antibodies 21F12 (recognizing Aβ42, but not Aβ40), 2G3 (recognizing Aβ40, but not Aβ42), 4G8 (binding Aβ between 13 and 28), 10D5 (recognizing 1-16), and 3D6 (binding 1-5) are administered to transgenic PDAPP mice as described above.

The performance of the mice administered these antibodies is then determined in the object recognition test as described above. Performance will correlate positively with the affinity of the antibody of soluble Aβ, that is, the higher the affinity of an antibody for soluble Aβ, the generally higher will be the performance in tests of cognition within a short time after administering the antibody.

Antibody m266 causes more significant flux of Aβ into the plasma and faster, more complete recovery of object recognition than does an antibody such as 3D6, which has an affinity for soluble Aβ that is about 1,000-fold less than that of m266. Antibodies having higher binding affinity for soluble Aβ will cause more pronounced flux and significantly faster and better improvement in cognitive function.

### Example 3

### Spatial Learning in APPV717F Mice Following a Single anti-Aβ Antibody Treatment

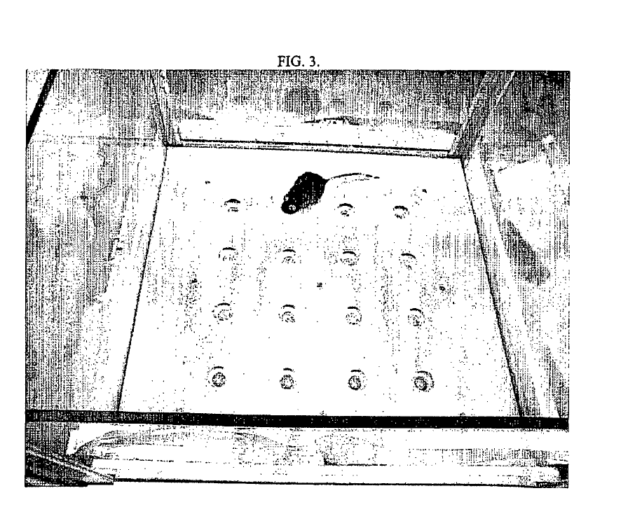

The subjects were female APPV717F and wild-type mice approximately 11 months old. Each mouse was administered 355 μg of murine 266 antibody or vehicle (PBS) administered 24 hours prior to start of testing (i.p.) Mice were tested in a holeboard spatial learning assay for four consecutive days (FIG. 3). Four holes were baited with access to a single food pellet and the remaining holes were baited beneath a screen without access. Mice were food-deprived each night before testing the next morning. Mice were tested for four, 180-second trials per day. Testing occurred for four consecutive days.

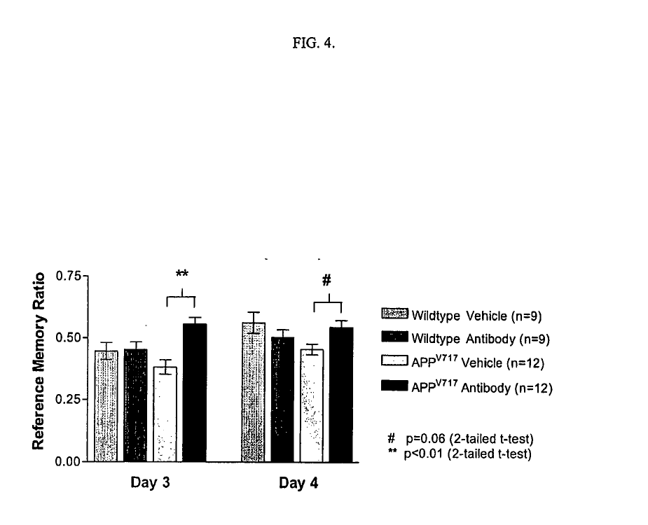

A single dose of Aβ antibody (266) significantly enhanced cognitive functioning of 11 month-old APPV717F mice compared to vehicle-treated transgenic mice (FIG. 4).

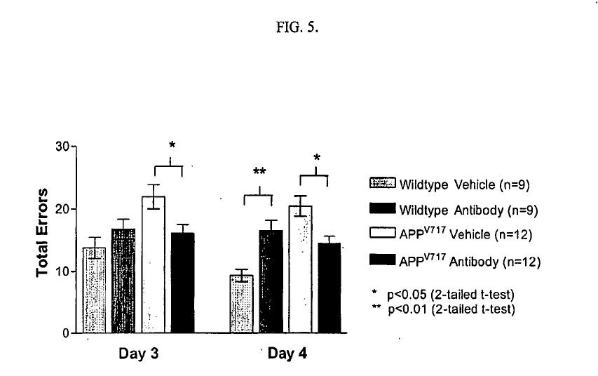

A significant decrease in total errors was noted on Day 4 for vehicle-treated WT mice while the number of errors was similar across Days 3 and 4 for antibody-treated wild type mice (FIG. 5).

### Example 4

### Effect of Administration of Various Antibodies on Plasma and Cortical Soluble Aβ Concentrations after 24 Hours

Transgenic (+/−) mice (4 months of age) were administered 355 tg of each antibody (intraperitoneal). Samples were obtained 24 hours later.

TABLE 1
Concentration of soluble Aβ42 in cortex (μg/g) 24 hours
after administration of various anti-Aβ antibodies.

| | Mean | S.E.M. |
|---|---|---|
| Control (5) | 0.058 | 0.007 |
| 266 (4) | 0.169 | 0.047 |
| 3D6 (5) | 0.091 | 0.007 |
| 10D5 (5) | 0.065 | 0.004 |

TABLE 2
Concentration (ng/mL) of Aβ in plasma samples
24 hours after administering various anti-Aβ antibodies.

| | |
|---|---|
| Aβ40 | Aβ42 |

| | Mean | S.E.M. | Mean | S.E.M. |
|---|---|---|---|---|
| Control (4) | 0.054 | 0.0045 | 0.064 | 0.004 |
| 266 (4) | 5.0 | 0.13 | 9.2 | 0.055 |
| 10D5 (5) | 0.19 | 0.022 | 0.20 | 0.002 |
| Control (4) | 0.50 | 0.02 | 0.13 | 0.005 |
| 3D6 (5) | 3.0 | 0.35 | 1.18 | 0.15 |

Antibody 3D6 administration caused an increase in plasma Aβ40 as well as Aβ42 (6-fold, and 8-fold, respectively). Plasma Aβ40 and Aβ42 levels were increased by 10D5 administration as well (approx. 3-4 fold). Antibody 266 administration caused a very significant increase in both Aβ40 and Aβ42 (93-fold and 144-fold, respectively).

Soluble Aβ40 from cortical tissue was significantly increased by 266 only. Administration of 3D6 or 10D5 was without effect on soluble Aβ40 levels in brain.

In another study of the rapid effects of administration of antibodies, 355 μg of each of 266, 3D6, and 4GS was administered ip to hemizygous PDAPP transgenic mice (3 months old). Samples were obtained 24 hours later.

TABLE 3
Concentration (ng/mL) of Aβ42 in plasma samples
24 hours after administering various anti-Aβ antibodies.

Aβ42

| | Mean | S.E.M. |
|---|---|---|
| Control (7) | 0.048 | 0.0018 |
| 266 (5) | 3.8 | 0.30 |
| 4G8 (5) | 0.23 | 0.035 |
| Control (7) | 0.088 | 0.0035 |
| 3D6 (5) | 0.72 | 0.15 |

In another study of the rapid effects of administration of antibodies, 355 μg of each of anti-Aβ antibodies 2G3 (recognizing the C-terminus of Aβ40 but not Aβ42), 10D5 (recognizing the N-terminus of Aβ), and 21F12 (recognizing the C-terminus of Aβ42, but not Aβ40) was administered intraperitoneally. Samples were obtained 24 hours later. The only significant difference in plasma Aβ42 levels was in the 10D5 group.

TABLE 4
Concentration (ng/mL) of Aβ42 in plasma samples
24 hours after administering various anti-Aβ antibodies.

Aβ42

| | Mean | S.E.M. |
|---|---|---|
| Control (5) | 0.065 | 0.0055 |
| 2G3 (5) | 2.34 | 0.091 |
| 10D5 (5) | 0.087 | 0.0044 |
| Control (5) | 0.058 | 0.0029 |
| 21F12 (5) | 1.80 | 0.036 |

TABLE 5
Concentration (μg/g) of soluble Aβ40 and Aβ42 in
cortex 24 hours after administering various anti-Aβ antibodies.

| | |
|---|---|
| Aβ40 | Aβ42 |

| | Mean | S.E.M. | Mean | S.E.M. |
|---|---|---|---|---|
| Control (5) | 27 | 3 | 7.0 | 0.61 |
| 2G3 (5) | 48 | 5 | 4.0 | 0.23 |
| 10D5 (5) | 62 | 9 | 5.2 | 0.49 |
| 21F12 (5) | 91 | 14 | 5.4 | 0.16 |

There were no significant differences in insoluble Aβ in any group. Administration of 21F12 and 10D5 resulted in a statistically significant increase in soluble Aβ40 levels in the brain. Soluble levels of Aβ42 were significantly decreased following treatment with all 3 antibodies.

In yet another study, administration of 360 μg of 266, 3D6, or 10D5 antibody per animal (5 animals per group, saline control) raised average plasma Aβ 1-40 approximately 334-, 92-, and 14-fold, respectively, and raised average plasma Aβ 1-42 approximately 168-, 32-, and 19-fold, respectively. Clearly the relative magnitudes of these responses are directly related to the relative affinities of the antibodies toward soluble Aβ.

Taken together, these studies of the effects of administration of anti-Aβ antibodies after 24 hours shows a strong relationship between the levels of plasma Aβ and the affinity of the antibody for soluble Aβ40 and Aβ42. Also, an antibody that causes a significantly higher flux of Aβ (i.e., 266 compared with 3D6) after 24 hours also causes a significantly higher recovery of cognition in the same period. Therefore, we believe that improvement in cognition will be faster and/or greater in magnitude when an antibody having a higher affinity for soluble Aβ is administered.

### Example 5

### Cognition after Administration of Antibodies Having a Range of Affinities for Soluble Aβ

Antibodies having affinities for soluble Aβ between about 1 nM and about 1 pM are obtained or prepared as described herein. The antibodies are administered to transgenic mice as described above in Example 1. Antibodies having higher affinity for soluble Aβ will generally cause greater flux of Aβ within a short time after administration and also more greatly effect rapid improvement in cognition.

### Example 6

### Binding Affinity for Soluble Aβ

Antibody affinity for soluble Aβ is determined using a BIAcore biosensor 2000 and data analyzed with BIAevaluation (v. 3.1) software. A capture antibody (rabbit anti-mouse Ig or anti-human Ig) is coupled via free amine groups to carboxyl groups on flow cell 2 of a biosensor chip (CM5) using N-ethyl-N-dimethylaminopropyl carbodiimide and N-hydroxysuccinimide (EDC/NHS). A non-specific rabbit IgG is coupled to flow cell 1 as a background control. Test antibodies are captured to yield 300 resonance units (RU). Soluble Aβ 1-40 or 1-42 (Biosource International, Inc.) is then flowed over the chip at decreasing concentrations (1000 to 0.1 times KD). To regenerate the chip, bound anti-Aβ antibody is eluted from the chip using a wash with glycine-HCl (pH 2). A control injection containing no amyloid-beta serves as a control for baseline subtraction. Sensorgrams demonstrating association and dissociation phases are analyzed to determine kd and ka. Using this method, the affinity of the following antibodies was determined for 1-42 and/or 1-40, and they are presented in Table 1. Two affinities were found for antibody 10D5.

TABLE 6
Affinity (nM) of various antibodies for soluble
Aβ1-40 and/or soluble Aβ1-42.

| | Soluble Aβ1-40 | Soluble Aβ1-42 |
|---|---|---|
| 3D6 | 2.4 | 2.4 |
| 10D5 | 390 | 0.57/4,950 |
| 266 | 0.004 | 0.004 |
| 266 | — | 0.0025 |
| N56S* | | |
| 266 | — | 0.0019 |
| N56T† | | |
| 4G8 | 23 | 24 |
| 21F12 | — | 4.4 |
| 2G3 | 0.9 | — |

*deglycosylated 266 analog, wherein Asn at position 56 of the heavy chain variable region is replaced with Ser
†deglycosylated 266 analog, wherein Asn at position 56 of the heavy chain variable region is replaced with Thr

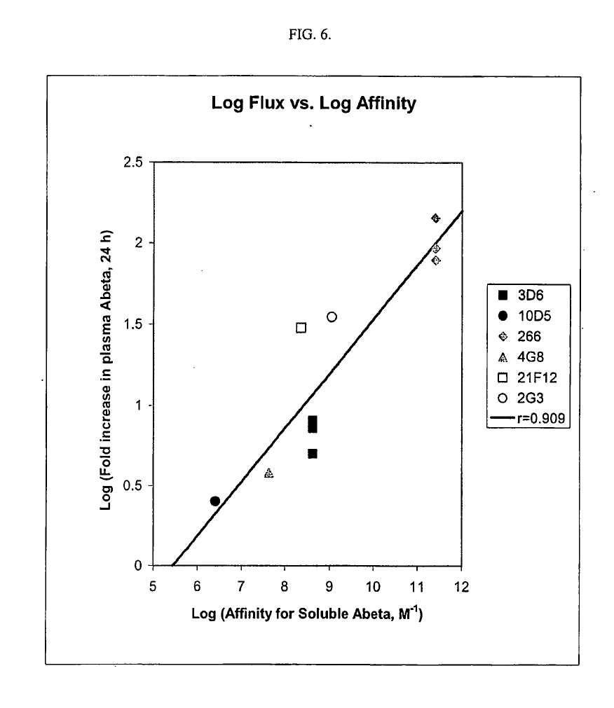

In FIG. 6 is plotted log of flux vs. log affinity for soluble Aβ. Flux is defined as the fold increase in plasma Aβ (40 or 42) 24 hours after administration of antibody, as described in Example 4 above. Affinity is given in Table 2 above. Because 10D5 apparently had two affinities for soluble Aβ42 that varied in the extreme, only data on Aβ40 were used for 10D5. A distinct relationship between affinity for soluble Aβ and flux is evident.

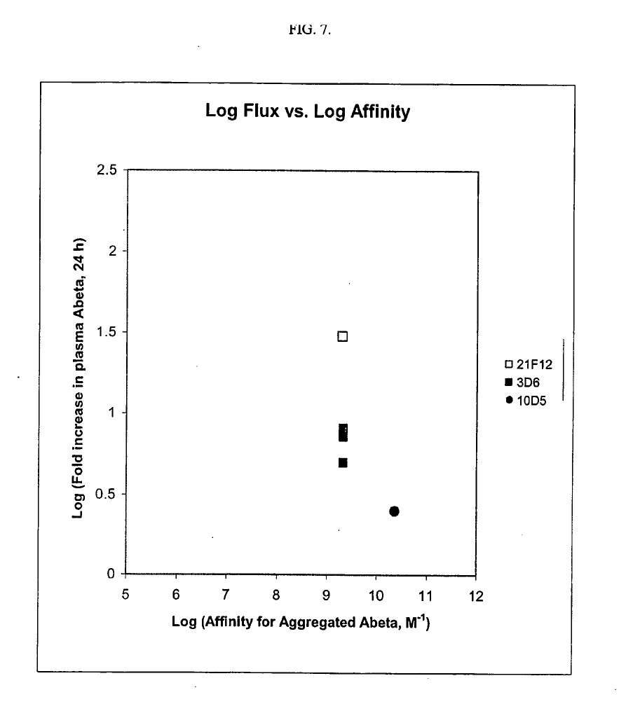

In FIG. 7 is plotted flux data against affinity for aggregated Aβ, using data taken from Bard, et al., Nat. Med. 6:916-919 (2000). Antibody 266 was reported not to bind to aggregated Aβ, and so no data are plotted for it. There is clearly no relationship between affinity for aggregated Aβ and flux.

As demonstrated herein, the affinity of anti-Aβ antibodies for soluble, not aggregated, Aβ is positively correlated with flux of Aβ from the brain into the plasma within 24 hours after administering the antibody. Furthermore, the rate of flux of Aβ is also related to acute improvement in cognitive performance as demonstrated in Example 1 and FIG. 2. Antibodies having higher affinity for soluble Aβ will cause more pronounced flux and will more quickly and more significantly effect improvement in cognitive function in conditions and diseases involving Aβ. Furthermore, chronic administration of antibodies having high affinity for soluble forms of Aβ will more effectively cause flux of Aβ from brain and sequester Aβ forms away from brain tissues which are adversely affected in conditions and diseases related to Aβ. We conclusively demonstrate here that high affinity for soluble, not insoluble or aggregated, forms of Aβ is an important feature of anti-Aβ antibodies for treating cognitive impairment in Aβ related conditions and diseases. We also believe that high affinity for soluble, not insoluble or aggregated, forms of Aβ is an important feature of anti-Aβ antibodies for treating cognitive impairment in Aβ related conditions and diseases.

### Example 7

### Alternate Procedure for Determining Binding Affinity for Antibodies Having High Affinity for Soluble Aβ

The procedure is very similar to that used above in Example 6, with certain modifications as described below. Protein A or protein A/G is immobilized via amine coupling to flow cells 1 and 2 of a B1 or CM5 sensor chip (BIAcore). The test antibody is then captured in flow cell 2 at desired levels (usually a 10-60 second injection of antibody) and 5 minutes is allowed for the antibody to stabilize. An aliquot of frozen Aβ 1-40 solution is thawed and then diluted to make up the highest concentration (e.g., 200 nM), which is then serially diluted (1:2 dilutions) to the lowest concentration (e.g., 6.25 nM). Each concentration is injected over the surface for 5 minutes at a flow rate of 50 μL/min. To obtain an accurate measurement of off-rate for high affinity antibodies, the dissociation of Aβ 1-40 is followed for 5 hours. Aβ 1-40 and antibody are then eluted from both flow cells with a 40 second injection of glycine (pH 1.5). The signal is allowed to stabilize for 2 minutes before the next cycle. The data from flow cell 1 is subtracted from flow cell 2 to account for any bulk shifts due to buffer differences or non-specific binding to the sensor chip or protein A. The various concentrations are injected randomly and each concentration is run in duplicate. Two 0 nM runs are used to control for any dissociation of antibody from the protein A or A/G capture surface. The data is analyzed using the Biaevalution software. A 1:1 model with mass transfer and a local Rmax is used as the best fit for the data.

Using this alternate method, the affinity of humanized 266 antibody for soluble Aβ 1-40 was found to be 0.4 M⁻⁹ (±0.2 M⁻⁹). Using this value of affinity, together with data for other antibodies (above), the log of flux vs. log affinity for soluble Aβ 1-40 was plotted (FIG. 8). The very distinct relationship between affinity for soluble Aβ and flux continues to be evident.

Soluble Aβ 1-40 stocks and diluted samples are prepared as follows. Solid Aβ 1-40 (Biosource International, Camarillo Calif. USA 93012) is dissolved to 1 mg/mL (about 230 μM) in water, and the solution is immediately aliquoted into 20-50 μL portions and then frozen (−70° C.). Alkaline conditions can be used to dissolve Aβ, as described by Fezoui, et al., Amyloid: Int'l J. Exp. Clin. Invest. 7:166-178 (2000). An alternate method for preparing aggregate-free soluble stock Aβ solutions is that of Zagorski, et al. [Meth. Enzymol. 309:189-204 (1999)]. This procedure involves, in sequence, dissolving the peptide in trifluoroacetic acid (TFA), evaporating the TFA, redissolving in hexafluoroisopropanol (HFIP), removing HFIP, and dissolving in water. Aliquoting and freezing may be performed either before or after removing HFIP. Stock Aβ solutions can be checked for aggregates by methods well known in the art, for example, light scattering [Tomski, et al., Arch. Biochem. Biophys. 294:630-638 (1992)], thioflavin T binding [LaVine, Meth. Enzymol. 309:274-285 (1999)], or Congo red binding [Klunk et al., Anal. Biochem. 266:66 (1999)]. Immediately before use, a stock aliquot is thawed and diluted to the highest concentration to be used (typically, about a thousand-fold dilution to about 200 nM). This 1000-fold diluted sample is expected to contain soluble Aβ that is predominantly monomeric. The great tendency of Aβ to self-associate means that even when carefully prepared as described, samples of Aβ may contain small proportions of Aβ dimer especially, and perhaps even some higher order association states. Stock aliquots are not refrozen after they have been thawed. Stock aliquots are not used after the day on which they are thawed, nor are diluted samples used after the day on which they are prepared.

### Example 8

### Effect of Administration of Various Humanized Antibodies on Plasma Aβ Concentrations in Male Cynomolgus Monkeys after 24 Hours

The animals (2 to 4 kg, young adult to adult, 2 animals per group) were given a bolus intravenous administration of antibody (1 mg/kg; humanized 266—expressed in a host cell transformed to express SEQ ID NO:11 and SEQ ID NO:12; N56S—expressed in a host cell transformed to express SEQ ID NO:11 and SEQ ID NO:21; or humanized 3D6—U.S. 60/287,539, filed 2001 Apr. 30). Animals were housed individually in stainless steel cages. Environmental controls were set to maintain 18 to 29 degrees Centigrade, a relative humidity of 30% to 70%, and a 12-hour light/12-hour dark cycle. They received certified primate diet one or two times daily. Water was provided ad libitum. The animals were selected for study based on body weights, clinical observations, clinical pathology data, and other data as appropriate. Plasma samples (pre-dose and 24 hours post-dose) were collected in EDTA and frozen until analysis.

Plasma concentrations of immunoreactive Aβ₁₋₄₀ or Aβ₁₋₄₂ were determined using ELISA assays. Immunoreactive Aβ₁₋₄₀ was captured on the ELISA plate using mouse monoclonal antibody 2G3 or Aβ₁₋₄₂ was captured using mouse monoclonal antibody 21F12. The bound complex was detected using biotinylated-3D6 antibody, followed by addition of streptavidin-HRP. Color development was performed using TMB as a substrate. Optical density values were read at 450/630 nm, and raw data was analyzed with a 5-parameter logistic algorithm using STATLIA software (Brendan Scientific). Serum concentrations of immunoreactive Aβ₁₋₄₀ were estimated using calibrators ranging from 16 to 1000 pg/ml prepared in heat-treated, charcoal stripped human serum. Based on recovery of control samples and the back-fit of calibrators, the lower and upper limits of quantitation in this assay are estimated to be 50 and 1000 pg/mL, respectively. Results for Aβ1-40 are shown below in Table 7. Flux could not be calculated for Aβ1-42 because pre-dose concentrations were below the detection limit.

TABLE 7
Mean concentrations of immunoreactive antibodies and
Aβ1-40 in cynomolgus monkeys (two animals per group).

| | humanized | | humanized |
|---|---|---|---|
| | 266 | N56S | 3D6 |
| [Aβ1-40]_{Pre-dose} (ng/mL) | 0.6 | 1.4 | 0.6 |
| [Aβ1-40]_{24 hour} (ng/mL) | 109 | 124 | 12.2 |
| Difference (ng/mL) | 108 | 123 | 11.6 |
| Flux (-fold increase) | 181 | 87.6 | 19.3 |

The increase in plasma Aβ1-40 24 hours after administration of humanized 266 and N56S was approximately the same, and much greater than the increase after administration of humanized 3D6. These data, obtained in normal monkeys (i.e., not having any known defect that affects Aβ metabolism or any known Aβ-related conditions or diseases), are consistent with the mouse studies described herein—that is, that the affinity of anti-Aβ antibodies for soluble, not aggregated, Aβ is positively correlated with flux of Aβ from the brain into the plasma within 24 hours after administering the antibody. N56S has a much faster turn-over than humanized 266 or 3D6 (5.5- and 8.6-times as much 266 and 3D6 in the plasma as N56S at 24 hours, though the doses were the same). Thus, the Aβ1-40 levels at 24 hours for N56S are higher than those for humanized 266 when normalized to the concentration of antibody present, as would be expected on the basis of the present invention, because N56S has a higher affinity for soluble Aβ than humanized 266. Differences in flux caused by 266 and 3D6 cannot be attributed to differences in the pharmacokinetics for the two antibodies either in these monkeys or in the mice used in other experiments described herein.

1-22. (cancelled).

23. A method for treating cognitive symptoms of a condition or disease associated with Aβ in a subject, comprising administering to the subject an effective amount of an anti-Aβ antibody that has affinity for soluble Aβ greater than 10⁻⁹ M.

24. The method of claim 23, wherein the anti-Aβ antibody has affinity for soluble Aβ greater than 10⁻¹⁰ M.

25. The method of claim 24, wherein the anti-Aβ antibody has affinity for soluble Aβ greater than 10⁻¹¹ M.

26. The method of claim 25, wherein the anti-Aβ antibody has affinity for soluble Aβ greater than 10⁻¹² M.

27. The method of claim 24, wherein the affinity of the anti-Aβ antibody is measured with respect to soluble Aβ 1-40 or Aβ 1-42.

28. The method of claim 24, wherein the anti-Aβ antibody recognizes the same epitope that antibody 266 recognizes or competes with antibody 266 for binding to soluble Aβ.

29. The method of claim 24, wherein the affinity of the anti-Aβ antibody for soluble Aβ is greater than the affinity of antibody 266 for soluble Aβ.

30. The method of claim 24, wherein the condition or disease is selected from the group consisting of Alzheimer's disease, Down's syndrome, cerebral amyloid angiopathy, vascular dementia, and mild cognitive impairment.

31. The method of claim 24, wherein the subject is human and the anti-Aβ antibody is a human or humanized antibody.

32. A method for reducing disease progression in a subject having a condition or disease associated with Aβ, comprising administering to the subject an effective amount of an anti-Aβ antibody that has affinity for soluble Aβ greater than 10⁻⁹ M.

33. The method of claim 32, wherein the anti-Aβ antibody has affinity for soluble Aβ greater than 10⁻¹⁰ M.

34. The method of claim 33, wherein the anti-Aβ antibody has affinity for soluble Aβ greater than 10⁻¹¹ M.

35. The method of claim 34, wherein the anti-Aβ antibody has affinity for soluble Aβ greater than 10⁻¹² M.

36. The method of claim 32, wherein the affinity of the anti-Aβ antibody is measured with respect to soluble Aβ 1-40 or Aβ 1-42.

37. The method of claim 32, wherein the anti-Aβ antibody recognizes the same epitope that antibody 266 recognizes or competes with antibody 266 for binding to soluble Aβ.

38. The method of claim 32, wherein the affinity of the anti-Aβ antibody for soluble Aβ is greater than the affinity of antibody 266 for soluble Aβ.

39. The method of claim 32, wherein the condition or disease is selected from the group consisting of Alzheimer's disease, Down's syndrome, cerebral amyloid angiopathy, vascular dementia, and mild cognitive impairment.

40. The method of claim 32, wherein the subject is human and the anti-Aβ antibody is a human or humanized antibody.


SEQUENCE LISTING (21 sequences)

SEQ ID NO: 1 - protein, 16 aa, Mus sp.
  MISC_FEATURE (1)..(16): Light Chain CDR1
RSSQSLIYSDGNAYLH

SEQ ID NO: 2 - protein, 7 aa, Mus sp.
  MISC_FEATURE (1)..(7): Light Chain CDR2
KVSNRFS

SEQ ID NO: 3 - protein, 9 aa, Mus sp.
  MISC_FEATURE (1)..(9): Light Chain CDR3
SQSTHVPWT

SEQ ID NO: 4 - protein, 5 aa, Mus sp.
  MISC_FEATURE (1)..(5): Heavy Chain CDR1
RYSMS

SEQ ID NO: 5 - protein, 17 aa, Mus sp.
  MISC_FEATURE (1)..(17): Heavy Chain CDR2
QINSVGNSTYYPDTVKG

SEQ ID NO: 6 - protein, 3 aa, Mus sp.
  MISC_FEATURE (1)..(3): Heavy Chain CDR3
GDY

SEQ ID NO: 7 - protein, 113 aa, Artificial
  Synthetic construct
DXVMTQXPLSLPVXXGQPASISCRSSQSLXYSDGNAYLHWFLQKPGQSPXLLIYKVSNRFSGVPDRFSGSGSGTDFTLKISRVEAEDXGVYYCSQSTHVPWTFGXGTXXEIKR

SEQ ID NO: 8 - protein, 112 aa, Artificial
  Synthetic construct
XVQLVEXGGGLVQPGGSLRLSCAASGFTFSRYSMSWVRQAPGKGLXLVAQINSVGNSTYYPDXVKGRFTISRDNXXNTLYLQMNSLRAXDTAVYYCASGDYWGQGTXVTVSS

SEQ ID NO: 9 - protein, 113 aa, Artificial
  Synthetic construct
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVPWTFGQGTKVEIKR

SEQ ID NO: 10 - protein, 112 aa, Artificial
  Synthetic construct
EVQLVESGGGLVQPGGSLRLSCAASGFTFSRYSMSWVRQAPGKGLELVAQINSVGNSTYYPDTVKGRFTISRDNAKNTLYLQMNSLRAEDTAVYYCASGDYWGQGTLVTVSS

SEQ ID NO: 11 - protein, 219 aa, Artificial
  Synthetic construct
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 442 aa, Artificial
  Synthetic construct
EVQLVESGGGLVQPGGSLRLSCAASGFTFSRYSMSWVRQAPGKGLELVAQINSVGNSTYYPDTVKGRFTISRDNAKNTLYLQMNSLRAEDTAVYYCASGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 17 aa, Mus sp.
  MISC_FEATURE (1)..(17): Heavy chain CDR2
QINSVGXXXYYPDTVKG

SEQ ID NO: 14 - protein, 17 aa, Mus sp.
  MISC_FEATURE (1)..(17): Heavy chain CDR2
QINSVGXXXYYPDTVKG

SEQ ID NO: 15 - protein, 17 aa, Mus sp.
  MISC_FEATURE (7)..(7): Xaa at position 7 is Asn
QINSVGXXXYYPDTVKG

SEQ ID NO: 16 - protein, 17 aa, Mus sp.
  MISC_FEATURE (7)..(7): Xaa at position 7 is Asn
QINSVGXXXYYPDTVKG

SEQ ID NO: 17 - protein, 17 aa, Mus sp.
  MISC_FEATURE (1)..(17): Heavy Chain CDR2
QINSVGXXXYYPDTVKG

SEQ ID NO: 18 - protein, 17 aa, Mus sp.
  MISC_FEATURE (7)..(7): Xaa at position 7 is Asn
QINSVGXXXYYPDTVKG

SEQ ID NO: 19 - protein, 112 aa, Artificial
  synthetic construct
XVQLVEXGGGLVQPGGSLRLSCAASGFTFSRYSMSWVRQAPGKGLXLVAQINSVGXXXYYPDXVKGRFTISRDNXXNTLYLQMNSLRAXDTAVYYCASGDYWGQGTXVTVSS

SEQ ID NO: 20 - protein, 112 aa, Artificial
  Synthetic construct
EVQLVESGGGLVQPGGSLRLSCAASGFTFSRYSMSWVRQAPGKGLELVAQINSVGXXXYYPDTVKGRFTISRDNAKNTLYLQMNSLRAEDTAVYYCASGDYWGQGTLVTVSS

SEQ ID NO: 21 - protein, 442 aa, Artificial
  Synthetic construct
EVQLVESGGGLVQPGGSLRLSCAASGFTFSRYSMSWVRQAPGKGLELVAQINSVGXXXYYPDTVKGRFTISRDNAKNTLYLQMNSLRAEDTAVYYCASGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK